(19) United States
(12) Patent Application Publication
(10) Pub. No.: US 2014/0336220 A1
(43) Pub. Date: Nov. 13, 2014

(54) COMPOSITION FOR PREVENTING OR TREATING OBESITY COMPRISING REBAMIPIDE

(71) Applicant: The Catholic University of Korea Industry-Academic Cooperation Foundation, Seoul (KR)

(72) Inventors: Mi-La Cho, Seoul (KR); Jun-Ki Min, Bucheon-si (KR); Joo-Yeon Jhun, Seoul (KR); Jae-Kyeong Byun, Jecheon-si (KR); Hye-Jwa Oh, Seoul (KR)

(21) Appl. No.: 14/256,708

(22) Filed: Apr. 18, 2014

Related U.S. Application Data
(63) Continuation-in-part of application No. PCT/KR12/08674, filed on Oct. 22, 2012.

(30) Foreign Application Priority Data
Oct. 21, 2011 (KR) 10-2011-0107914

(51) Int. Cl. A61K 31/4704

(52) U.S. Cl. CPC A61K 31/4704
USPC 514/312

(57) ABSTRACT

The present disclosure relates to a composition and a health functional food for preventing or treating obesity comprising a rebamipide compound or its pharmaceutically acceptable salt as an active component. It is confirmed that when a rebamipide compound of the present disclosure is administered to a mouse model induced with obesity, it shows excellent effects of reducing weight, reducing adipocyte, and reducing a total cholesterol content in the body, as compared with a non-administered control group, and also, it shows an excellent effect of suppressing differentiation of cytotoxic Th17 cells that generate and secrets inflammatory cytokine and an excellent effect of improving activity of regulatory T cells (Treg) capable of suppressing a function of abnormally activated immune cells and controlling an inflammatory reaction. Thus, the rebamipide compound can be used for producing a medicine and a functional food which can effectively treat obesity caused by abnormality of immune modulation.

### CROSS-REFERENCE TO RELATED APPLICATIONS

This application is based on and claims priority from Korean Patent Application No. 10-2011-0107914, filed on Oct. 21, 2011, with the Korean Intellectual Property Office, the disclosure of which is incorporated herein in its entirety by reference.

### TECHNICAL FIELD

The present disclosure relates to a composition for preventing or treating obesity comprising rebamipide, and particularly, to a composition for preventing or treating obesity comprising rebamipide as an active component capable of preventing or treating obesity caused by abnormality of immune response and complicated interactions between genetic, metabolic, and environmental factors.

### BACKGROUND

Obesity is a severe chronic syndrome characterized by excessive accumulation of fat with various causes. There are two aims of treatment for obesity. A first aim is to reduce weight by burning excessive fat, and a second aim is to improve a metabolic imbalance. Patients with abdominal obesity are often relevant to diseased conditions such X-syndrome (insulin resistant diabetes, Type 2 diabetes, hypertension, and disorders of lipid metabolism), and abdominal obesity becomes one of the potent risk factors of early arteriosclerosis, ischemic heart disease, and cerebrovascular disease.

It is known that genetic predispositions account for more than 70% of causes of obesity, and other environmental factors include intake of high fat diet or lack of exercise. In recent years, abnormality of immune response is also considered as a cause of obesity.

T cells play a central role in the immune system as a host defense system against various pathogens. The T cells originate from the thymus of the human body and develop into T cells with unique properties through a series of differentiation processes. The differentiated T-cells are largely classified into Type 1 helper T cells (Th1) and Type 2 helper T cells (Th2) depending on a function. To be specific, a main function of the Th1 cells is involved in cell-mediated immunity, and a main function of the Th2 cells is involved in humoral immunity. In the immune system, these two cell groups maintain a balance through mutual control so that both are not excessively activated.

Therefore, it can be assumed that most of immune diseases are caused by an imbalance between these two immune cells. For example, it is known that abnormal increase of Th1 cell activity can lead to autoimmune disease, and abnormal increase of Th2 cell activity can lead to immune disease due to hypersensitive reaction.

Meanwhile, recent study on Th1 cell differentiation has reported the presence of regulatory T cells (Treg) as a new group that can regulate Th1 cell activity, and following this, many studies have introduced treatment for immune diseases using the regulatory T cells. Since the Treg cells are characterized by inhibiting functions of abnormally activated immune cells to thus control an inflammatory reaction, many studies report about experiments designed to treat immune diseases by the mechanism of increasing Treg cell activity.

In addition to Treg cells, Th17 cells are another group generated in a differentiation process. It is known that Th17 cells are developed from a differentiation process of non-differentiated T cells through a similar process as Treg cells. That is, differentiation of both Treg cells and Th17 cells commonly occurs in the presence of TGF-β. However, while Treg cells do not require IL-6, Th17 cells are differentiated in the presence of TGF-β together with IL-6. Further, the differentiated Th17 cells secrete IL-17.

Meanwhile, as diet pills currently used, a fat absorption inhibitor such as Xenical of Roche, Switzerland and an amphetamie such as Meridia of Abbott, U.S.A. have been usually used. However, such medicines have a problem of side effects such as headache, rise of blood pressure, diarrhea, or the like.

Thus, development of a new diet pill which is cheap and has an excellent treatment effect without side effects has been needed.

### SUMMARY

Thus, the inventors of the present disclosure completed the present disclosure based on the finding that among animal models induced with obesity, a group treated with rebamipide induces an effect of reducing weight and also reduces a total cholesterol content and an LDL-cholesterol content but increases a content of HDL-cholesterol useful in the body, as compared with a non-treated group, and, thus, can prevent or treat obesity.

Therefore, an object of the present disclosure is to provide a composition for preventing or treating obesity comprising a rebamipide compound or its pharmaceutically acceptable salt as an active component.

Another object of the present disclosure is to provide a composition comprising a rebamipide compound or its pharmaceutically acceptable salt as an active component for preventing or treating complications caused by obesity selected from the group consisting of intra-abdominal fat syndrome, metabolic syndrome, hypertriglyceridemia, hypo-HDL cholesterolemia, angina, myocardial infarction, ostarthritis, cancers related to a weight gain, orthostatic hypotension, pulmonary hypertension, diabetes, hypertension, damaged glucose tolerance, coronary thrombosis, atherosclerosis, gall bladder diseases such as cholelithiasis, insulin resistant diabetes, chronic total occlusion, thromboembolism, heart diseases, lipid syndrome, and hyperglycemia.

Further, yet another object of the present disclosure is to provide a f health functional food for preventing or improving obesity comprising a rebamipide compound or its salt as an active component.

Besides, still another object of the present disclosure is to provide a health functional food comprising a rebamipide compound or its pharmaceutically acceptable salt as an active component for preventing or improving complications caused by obesity selected from the group consisting of intra-abdominal fat syndrome, metabolic syndrome, hypertriglyceridemia, hypo-HDL cholesterolemia, angina, myocardial infarction, ostarthritis, cancers related to a weight gain, orthostatic hypotension, pulmonary hypertension, diabetes, hypertension, damaged glucose tolerance, coronary thrombosis, atherosclerosis, gall bladder diseases such as cholelithiasis, insulin resistant diabetes, chronic total occlusion, thromboembolism, heart diseases, lipid syndrome, and hyperglycemia.

In order to achieve the above-described objects, an exemplary embodiment of the present disclosure provides a composition for preventing or treating obesity comprising a rebamipide compound or its pharmaceutically acceptable salt as an active component. Otherwise, an exemplary embodiment of the present disclosure provides a use of a rebamipide compound or its pharmaceutically acceptable salt for preparing a composition for preventing or treating obesity. Still otherwise, an exemplary embodiment of the present disclosure provides a method for preventing or treating obesity comprising administering an effective dose of a rebamipide compound or its pharmaceutically acceptable salt to a subject in need of prevention or treatment of obesity.

Another exemplary embodiment of the present disclosure provides a use of a rebamipide compound or its pharmaceutically acceptable salt for preparing a composition for preventing or treating obesity.

In the exemplary embodiment of the present disclosure, the rebamipide has an effect of reducing weight, an effect of reducing adipocyte, and an effect of reducing total cholesterol, glucose, and LDL-cholesterol in the body.

In the exemplary embodiment of the present disclosure, the rebamipide has anti-obesity activity through conversion from white fat to brown fat.

In the exemplary embodiment of the present disclosure, the rebamipide can promote or increase activity or amplification of regulatory T cells (Treg).

In the exemplary embodiment of the present disclosure, the rebamipide can decrease or suppress differentiation of non-differentiated T cells to Th17 cells.

In the exemplary embodiment of the present disclosure, the rebamipide is contained at a concentration of 10 mg/kg to 1000 mg/kg.

Yet another exemplary embodiment of the present disclosure provides a composition comprising a rebamipide compound or its pharmaceutically acceptable salt as an active component for preventing or treating complications caused by obesity selected from the group consisting of intra-abdominal fat syndrome, metabolic syndrome, hypertriglyceridemia, hypo-HDL cholesterolemia, angina, myocardial infarction, ostarthritis, cancers related to a weight gain, orthostatic hypotension, pulmonary hypertension, diabetes, hypertension, damaged glucose tolerance, coronary thrombosis, atherosclerosis, gall bladder diseases such as cholelithiasis, insulin resistant diabetes, chronic total occlusion, thromboembolism, heart diseases, lipid syndrome, and hyperglycemia. Otherwise, yet another exemplary embodiment of the present disclosure provides a use of a rebamipide compound or its pharmaceutically acceptable salt for preparing a composition for preventing or treating the complications caused by obesity. Still otherwise, yet another exemplary embodiment of the present disclosure provides a method for preventing or treating the complications caused by obesity comprising administering an effective dose of a rebamipide compound or its pharmaceutically acceptable salt to a subject in need of prevention or treatment of the complications caused by obesity.

Still another exemplary embodiment of the present disclosure provides a health functional food for preventing or improving obesity comprising a rebamipide compound or its salt as an active component. Otherwise, still another exemplary embodiment of the present disclosure provides a use of a rebamipide compound or its pharmaceutically acceptable salt for preparing a health functional food for preventing or improving obesity. Still otherwise, still another exemplary embodiment of the present disclosure provides a method for preventing or improving the complications caused by obesity comprising administering an effective dose of a rebamipide compound or its pharmaceutically acceptable salt to a subject in need of prevention or improvement of obesity.

Further, further still another exemplary embodiment of the present disclosure provides a health functional food comprising a rebamipide compound or its pharmaceutically acceptable salt as an active component for preventing or improving complications caused by obesity selected from the group consisting of intra-abdominal fat syndrome, metabolic syndrome, hypertriglyceridemia, hypo-HDL cholesterolemia, angina, myocardial infarction, ostarthritis, cancers related to a weight gain, orthostatic hypotension, pulmonary hypertension, diabetes, hypertension, damaged glucose tolerance, coronary thrombosis, atherosclerosis, gall bladder diseases such as cholelithiasis, insulin resistant diabetes, chronic total occlusion, thromboembolism, heart diseases, lipid syndrome, and hyperglycemia. Otherwise, further still another exemplary embodiment of the present disclosure provides a use of a rebamipide compound or its pharmaceutically acceptable salt for preparing a health functional food for preventing or improving the complications caused by obesity. Still otherwise, further still another exemplary embodiment of the present disclosure provides a method for preventing or improving the complications caused by obesity comprising administering an effective dose of a rebamipide compound or its pharmaceutically acceptable salt to a subject in need of prevention or improvement of the complications caused by obesity.

According to the exemplary embodiments of the present disclosure, it is confirmed that when a rebamipide compound is administered to a mouse model induced with obesity, it shows excellent effects of reducing weight, reducing adipocyte, and reducing a total cholesterol content in the body, as compared with a non-administered control group, and also, it shows an excellent effect of suppressing differentiation of cytotoxic Th17 cells that generate and secretes inflammatory cytokine and an excellent effect of improving activity of regulatory T cells (Treg) capable of suppressing a function of abnormally activated immune cells and controlling an inflammatory reaction. Thus, it can be used for producing a medicine and a functional food which can effectively treat obesity caused by abnormality of immune modulation.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1A is a graph obtained by measuring a change in weight when rebamipide is orally administered to mice induced with obesity using a high fat food according to an exemplary embodiment of the present disclosure;

FIG. 1B illustrates a result of observation of livers (a and b) and mice (c and d) obtained by killing the mice on the 51st day after rebamipide is orally administered to the mice induced with obesity using a high fat food;

FIG. 2 illustrates a result of observation of an amount of adipocyte in a liver through tissue staining after rebamipide is orally administered to mice induced with obesity using a high fat food and the animal models are killed according to an exemplary embodiment of the present disclosure;

FIG. 3 is a graph obtained by measuring glucose, triglyceride, total cholesterol, LDL-cholesterol, and HDL-cholesterol from the serum obtained after rebamipide is orally administered to mice induced with obesity using a high fat food and the animal models are killed according to an exemplary embodiment of the present disclosure;

FIG. 4A illustrates a result of observation of a degree of expression of IL-17 in blood through flow cytometry after Th17 cells are induced to be differentiated by rebamipide according to an exemplary embodiment of the present disclosure;

FIG. 4B illustrates a result of observation of a degree of expression of IL-17 in spleen cells through flow cytometry after Th17 cells are induced to be differentiated by rebamipide according to an exemplary embodiment of the present disclosure;

FIG. 5A illustrates a result of observation of Th17 cells and Treg cells in spleen cells from animal models treated with rebamipide with a confocal microscope according to an exemplary embodiment of the present disclosure;

FIG. 5B is a graph obtained by measuring the number of Th17 cells and Treg cells in spleen cells from animal models treated with rebamipide with a confocal microscope according to an exemplary embodiment of the present disclosure;

FIG. 6A is a graph obtained by measuring a change in weight when rebamipide is orally administered with a dose of 30 mg/kg and 100 mg/kg to mice induced with obesity using a high fat food according to an exemplary embodiment of the present disclosure;

FIG. 6B illustrates a result of observation of livers and mice obtained by killing the mice on the 63rd day after rebamipide is orally administered to the mice induced with obesity using a high fat food;

FIG. 7A is a result of measurement of cholesterol, LDL-cholesterol, and triglyceride in the serum of an experimental animal according to an exemplary embodiment of the present disclosure;

FIG. 7B is a result of measurement of a degree of cell proliferation of mice of each group according to an exemplary embodiment of the present disclosure;

FIG. 8 is a result of measurement of an mast cell inhibition effect of rebamipide through Oil red O staining method according to an exemplary embodiment of the present disclosure;

FIGS. 9A and 9B show results of observation of an mast cell inhibition effect of rebamipide through real time PCR according to an exemplary embodiment of the present disclosure;

FIG. 10 is a result of measurement of amount of brown fat with respect to an amount of white fat by separating the brown fat and the white fat caused by a treatment with rebamipide and the white fat and weighing the brown fat and the white fat;

FIG. 11 is a result of measurement of an obesity induced arthritis index after a treatment with rebamipide;

FIG. 12 is a result of a histological inspection on animal models with collagen induced arthritis after a treatment with rebamipide according to an exemplary embodiment of the present disclosure;

FIG. 13 is a result of measurement on animal models with collagen induced arthritis by an ELISA method after a treatment with rebamipide according to an exemplary embodiment of the present disclosure;

FIG. 14 is a result of analysis with an optical microscope after animal models with obesity induced arthritis is stained by an immunochemical staining method in order to check whether rebamipide can inhibit inflammatory cytokine in the joints of the animal models;

FIG. 15 is a result of analysis on a Th17 cell inhibition effect and a Treg cell increase effect of rebamipide in models with obesity induced arthritis; and

FIG. 16 is a result of measurement of analysis of a STAT inhibition effect of rebamipide in models with obesity induced arthritis with a confocal microscope.

### DETAILED DESCRIPTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

In the present disclosure, it is first found that rebamipide has an effect of preventing or treating obesity, and the present disclosure provides a composition for preventing or treating obesity comprising a rebamipide compound or its pharmaceutically acceptable salt as an active component.

The inventors of the present disclosure have paid attention to a rebamipide compound while studying to develop a new diet pill for treating obesity. Rebamipide is a medicine which is highly effective in treating damage to the gastric mucous membrane caused by acute exacerbation of gastric ulcer, acute gastritis or chronic gastritis, and has been widely used as a medicine for treating peptic ulcer. Its chemical name is 2-(4-chlorobenzoylamino)-3-[2(1H)-quinolinon-4-yl]propionic acid.

This medicine promotes biosynthesis of PGE2 and increases mucus, and, thus, protects the gastric mucous membrane and promotes cell proliferation. In particular, as for a patient infected with Helicobacter pylori, this medicine suppresses adhesion and infiltration of Helicobacter pylori into gastric mucous membrane cells, thereby suppressing gastric mucosal inflammation.

However, conventionally, there has been no mention about a use of rebamipide for preventing or treating obesity.

Therefore, in the present disclosure, it is first found that a rebamipide compound can be used for preventing or treating obesity. In particular, it was confirmed that an obesity inhibition effect caused by administration of rebamipide is observed from animal models induced with obesity. Further, it was confirmed that rebamipide can suppress differentiation of Th17 cells as pathogenic cells and also promotes activity of regulatory T cells (Treg).

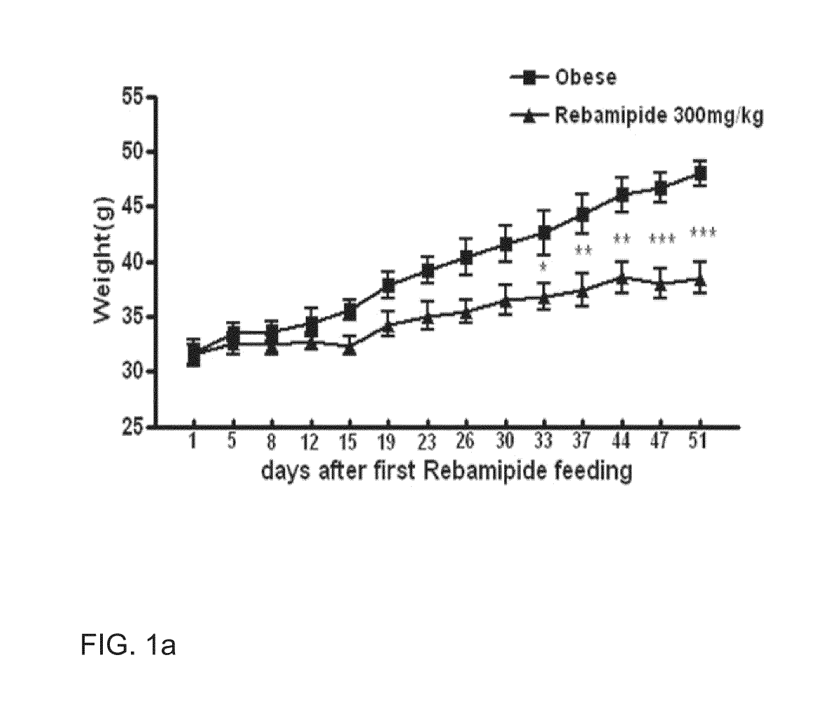

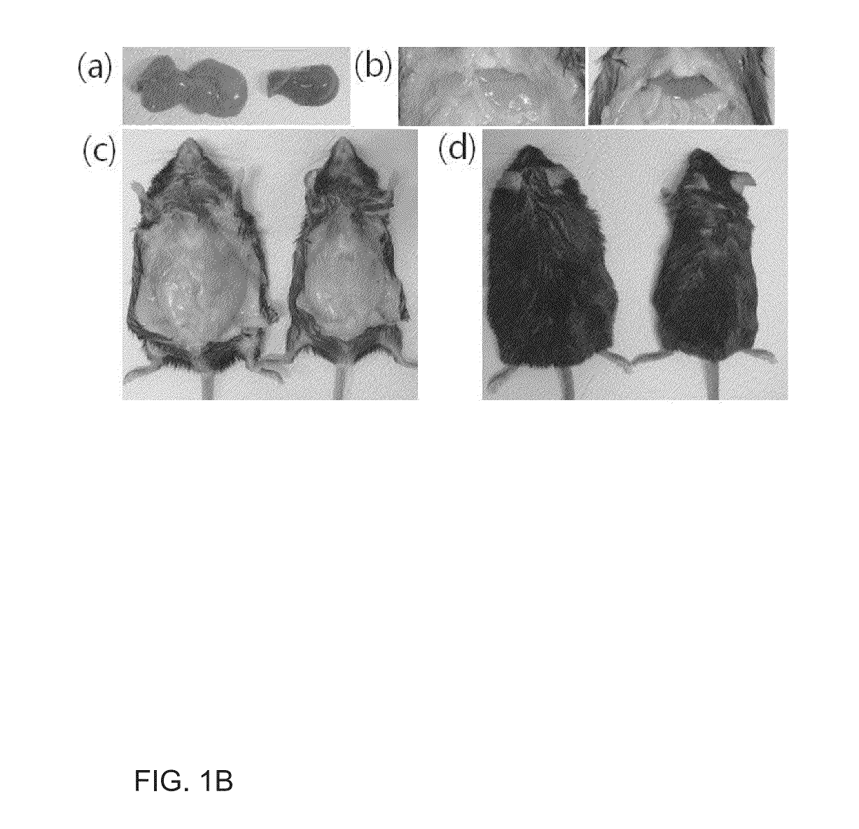

To be more specific, according to an exemplary embodiment of the present disclosure, it could be seen that when rebamipide was orally administered with a dose of 300 mg/kg to a mouse induced with obesity using a high fat food, a weight was statistically significantly decreased (refer to FIG. 1A). As a result of observation of shapes and livers of the obese mice, a liver of the mouse orally administered with rebamipide was bright red like a normal liver, whereas a liver of the control induced with obesity but not administered with rebamipide was yellow and the mouse was obese (refer to FIG. 1B).

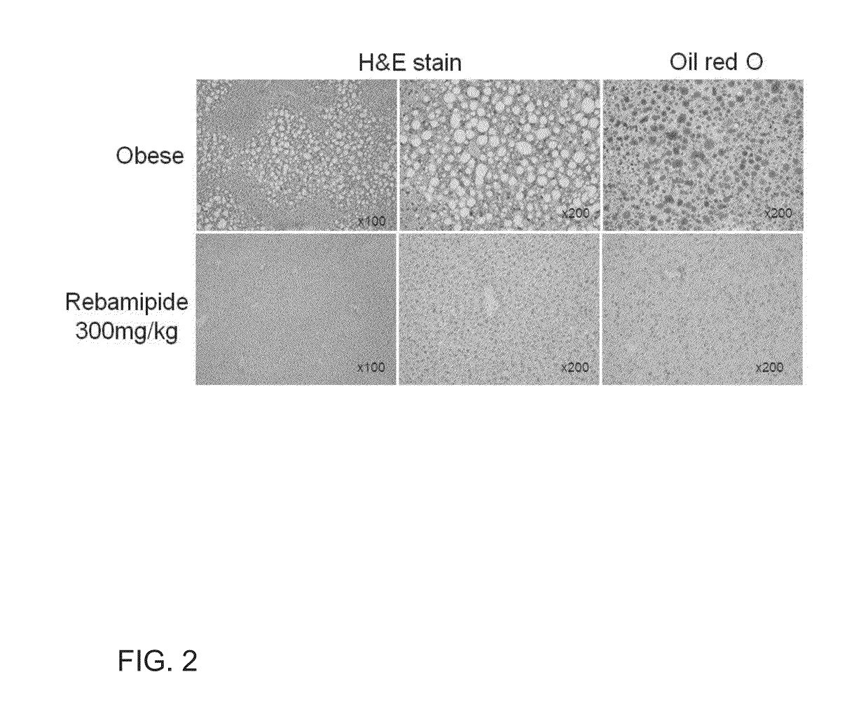

Further, according to another exemplary embodiment, in order to check whether a rebamipide compound has an effect of reducing adipocyte, the inventors of the present disclosure orally administered rebamipide to a mouse induced with obesity, killed the mouse on the 51st day after administration, and observed adipocyte from frozen sections of the liver of the mouse through H&E staining and Oil red O staining. As a result of the observation, it could be seen that adipocyte in a group treated with rebamipide was remarkably reduced as compared with those in the control (refer to FIG. 2).

Furthermore, in order to check whether the rebamipide compound has an effect of lowering a blood lipid concentration which may be a cause of arteriosclerosis, obesity, or the like, the inventors of the present disclosure measured glucose, triglyceride, total cholesterol, LDL-cholesterol, and HDL-cholesterol from the serum obtained from the killed mouse.

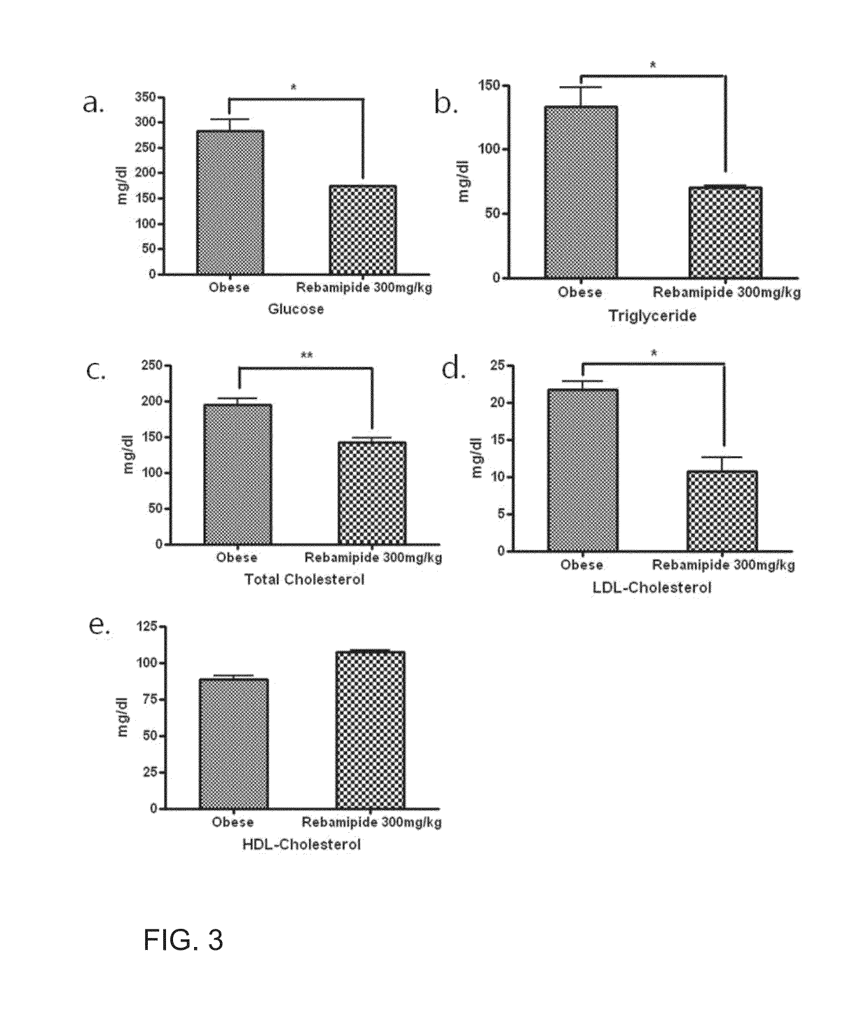

As a result of the measurement, it could be seen that in the serum of the mouse orally administered with rebamipide, glucose, triglyceride, total cholesterol, and LDL-cholesterol were statistically significantly decreased, whereas HDL-cholesterol useful in the body was not statistically significant but increased (refer to FIG. 3).

Therefore, according to this result, the inventors of the present disclosure found the fact that the rebamipide compound reduces a weigh of the mouse induced with obesity and reduces contents of the total cholesterol, triglyceride, and glucose in the body, thereby suppressing obesity.

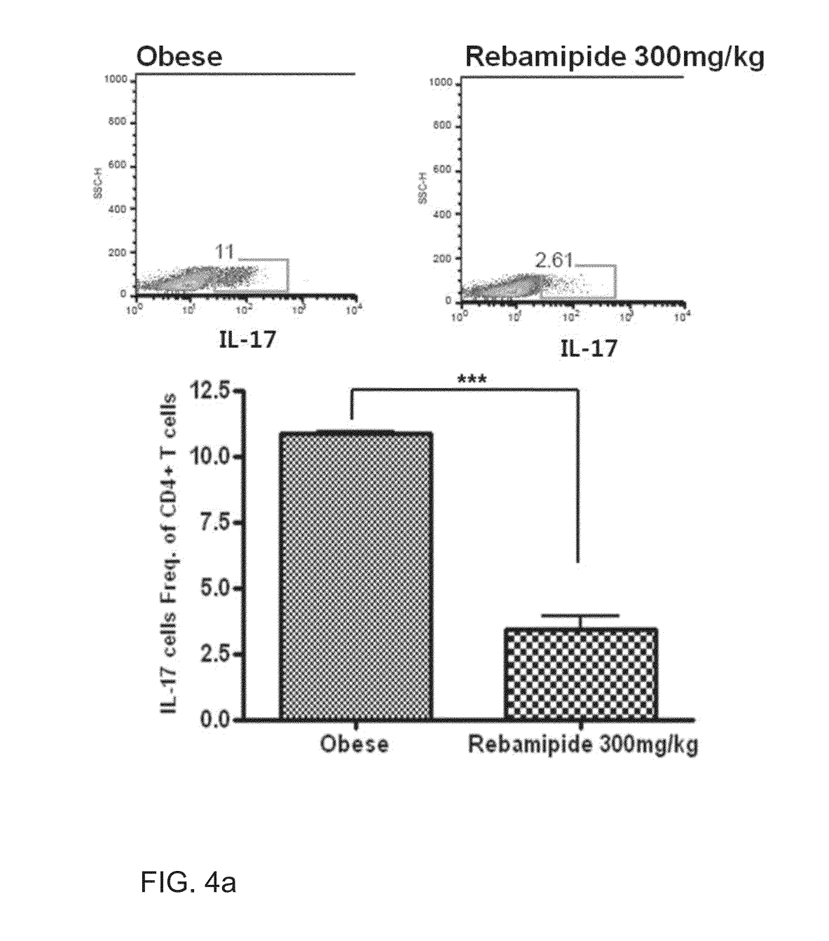

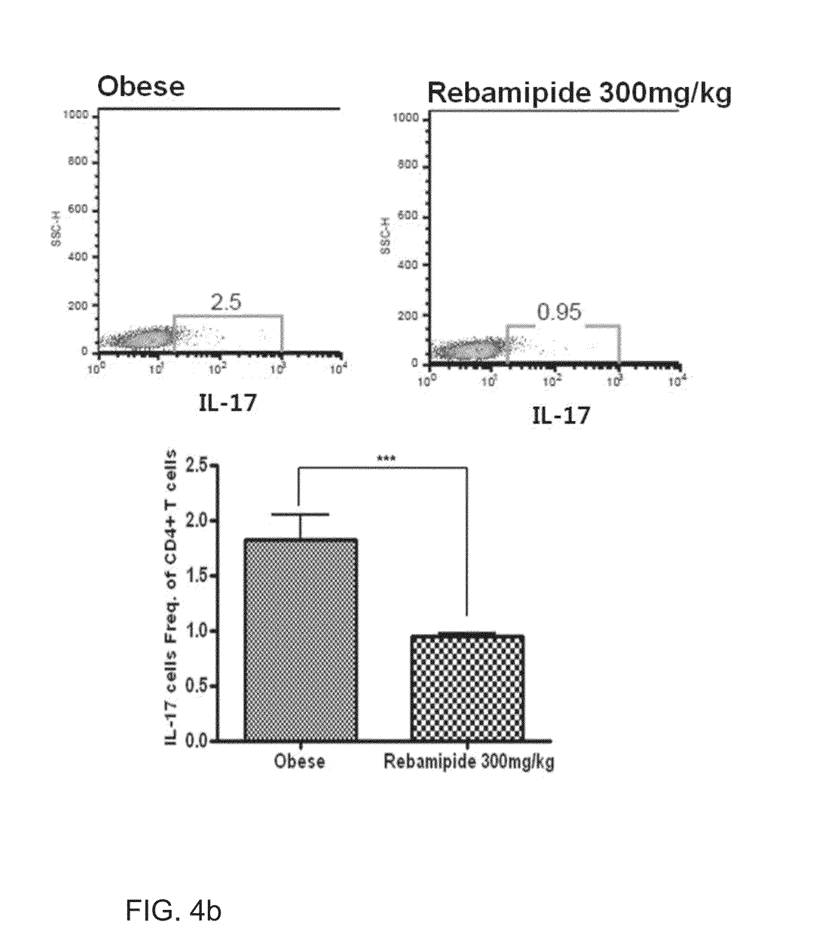

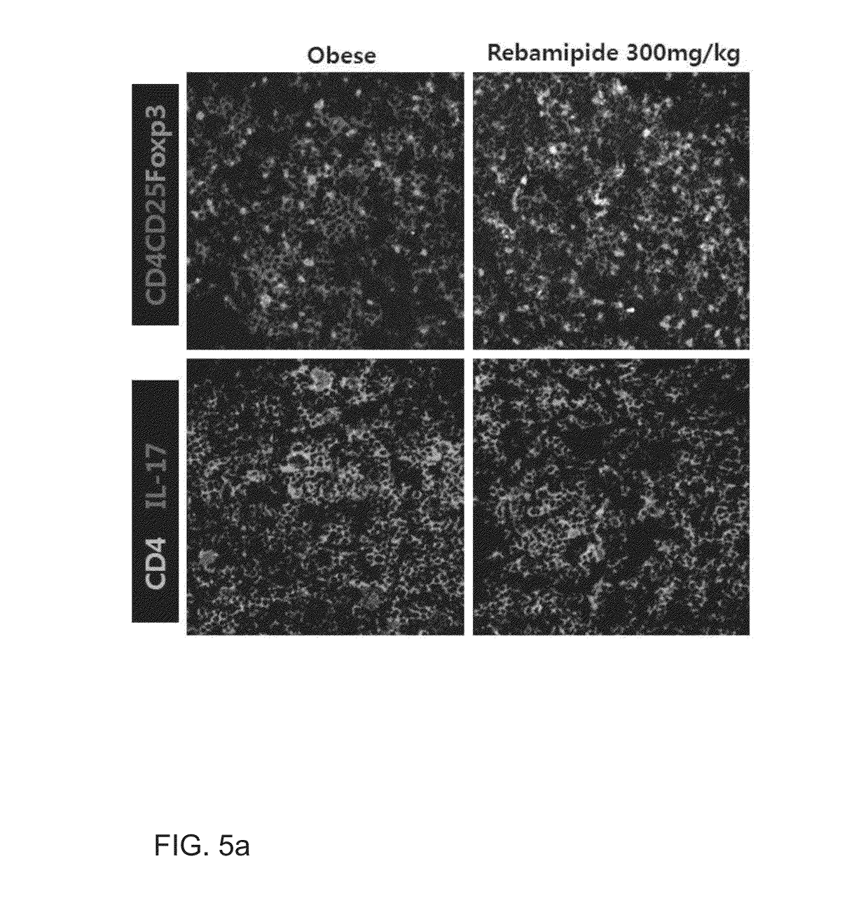

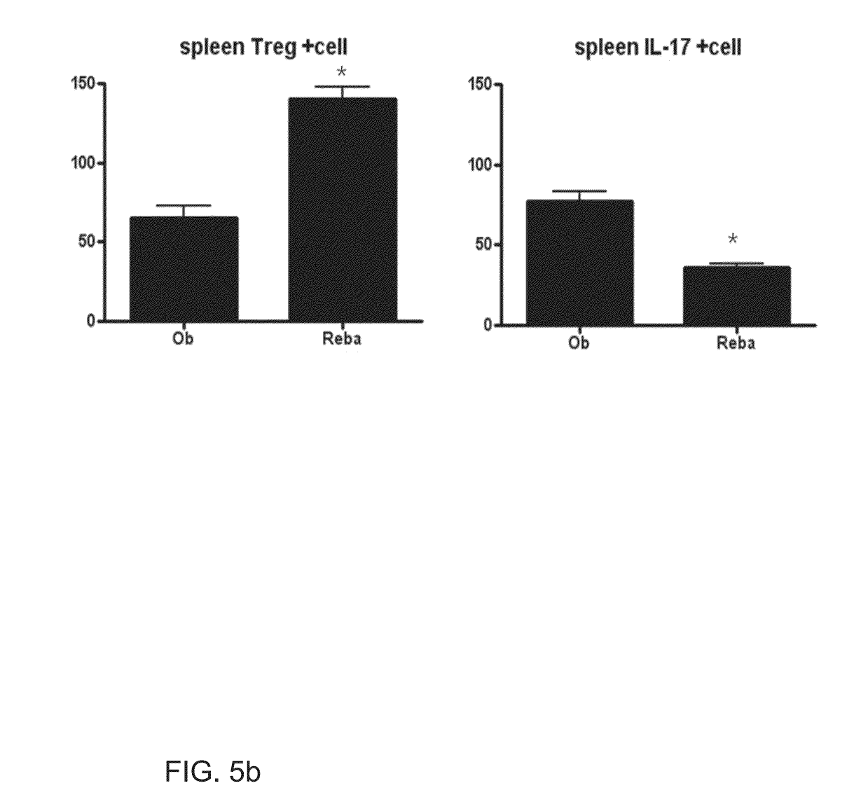

Moreover, the inventors of the present disclosure conducted a study on whether the rebamipide compound can suppress obesity of an animal model induced with obesity through an immune modulation. That is, according to an exemplary embodiment of the present disclosure, among mice induced with obesity, a group administered with rebamipide and a control mouse which was not administered with rebamipide were killed, and blood and spleen cells were obtained therefrom, and, then, Th17 cells as pathogenic cells that secrete inflammatory cytokine were observed from the blood and spleen cells using a flow cytometer. As a result of the observation, it could be seen that in the mouse treated with rebamipide, Th17 cells were significantly reduced in both the blood and the spleen cells (refer to FIG. 4 (a and b)), whereas regulatory T cells (Treg) were increased (refer to FIG. 5 (a and b)).

Therefore, according to this result, the inventors of the present disclosure found the fact that the rebamipide compound of the present disclosure can also prevent or treat obesity caused by abnormality of immune response. In the present disclosure, the compound “rebamipide” may include all forms of rebamipide, such as anhydrous forms, hydrate forms (for example, hemihyrate form), crystalline forms, or the like, and a pharmaceutically acceptable salt thereof. The rebamipide according to the present disclosure may be a compound represented by Chemical Formula 1 below.

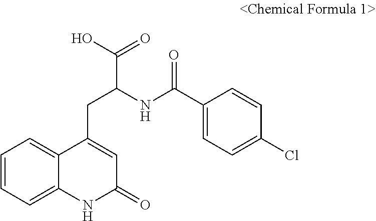 [SMILES: O=C(NC(Cc1cc(=O)[nH]c2ccccc12)C(=O)O)c1ccc(Cl)cc1]

Further, the pharmaceutically acceptable salt of the rebamipide includes an inorganic ionic salt originated from calcium, potassium, sodium, and magnesium; an inorganic acid salt originated from hydrochloric acid, nitric acid, phosphoric acid, bromic acid, iodic acid, and sulfuric acid; an organic acid salt originated from acetic acid, formic acid, succinic acid, tartaric acid, citric acid, trichloroacetic acid, trifluoroacetic acid, gluconic acid, benzoic acid, lactic acid, fumaric acid, and maleic acid; an sulfonic acid salt originated from methanesulfonic acid, ethanesulfonic acid, benzenesulfonic acid, p-toluenesulfonic acid, and naphthalenesulfonic acid; an amino acid salt originated from glycine, arginine, and lysine; and an amine salt originated from trimethylamine, triethylamine, ammonia, pyridine, and picoline.

A pharmaceutical composition of the present disclosure comprises a pharmaceutically acceptable carrier and can be formulated according to conventional methods into oral dosage forms such as powders, granules, tablets, capsules, suspensions, emulsions, syrups, or aerosols; external dosage forms; suppository; or sterile injection solution.

Preferably, the pharmaceutical composition of the present disclosure may be a form for oral administration, and more preferably, an oral solid dosage form of a tablet or capsule form. For example, the pharmaceutical composition of the present disclosure may be in the form of a commercially marketed rebamipide-containing tablet [for example, Mucosta Tablet (Otsuka Pharmaceutical Co., Ltd.)].

The pharmaceutically acceptable carrier includes lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, hydroxypropyl cellulose, low-substituted hydroxypropyl cellulose, hydroxypropyl methylcellulose 2910, polyethylene glycol 6000, polyvinylpyrrolidone, methyl hydroxybenzoate, propyl hydroxybenzoate, titanium dioxide, talc, magnesium stearate, mineral oil, or the like.

The pharmaceutical composition may further include a diluent or an excipient, such as filler, an extender, a binder, a humectant, a disintegrant, or a surfactant. A solid oral formulation includes a tablet, a pill, a powder, a granule, or a capsule. Such solid formulations may include at least one excipient selected from, for example, starch, calcium carbonate, sucrose or lactose, and gelatin. Such solid formulations may further include a lubricant, such as magnesium stearate or talc.

A liquid oral formulation includes a suspension, a solution, an emulsion, or syrup. In addition, the liquid oral formulation may include a diluent such as water, liquid paraffin, etc.; a humectant; a sweetening agent; an odorant; or a preservative. A parenteral formulation includes a sterile aqueous solution, a non-aqueous solution, a suspension, an emulsion, a lyophilized formulation, or a suppository. Non-aqueous solvents or suspensions include propylene glycol, polyethylene glycol, vegetable oil such as olive oil, or injectable esters such as ethyl oleate.

A dose of rebamipide contained in the pharmaceutical composition according to the present disclosure may vary depending on patient's state or body weight, seriousness of disease, dosage forms, administration routes, and the period of administration, and can be appropriately determined by one of ordinary skill in the art. For example, the rebamipide may be administered in a dose of 0.1 to 100 mg/kg, preferably 0.5 to 100 mg/kg, more preferably 0.5 to 5 mg/kg, per day. The administration can be carried out once or several times per day.

The pharmaceutical composition of the present disclosure can be also administered independently or in combination with other publicly-known compounds having an effect of preventing, improving, or treating symptoms of obesity. When administered as a combination, the therapeutic agents can be administered sequentially or at the same time.

Further, the present disclosure may provide a method for reducing or suppressing differentiation of non-differentiated T cells into Th17 cells in vitro, the method comprising treating the non-differentiated T cells with a rebamipide compound or its salt. The differentiation of non-differentiated T cells into Th17 cells can be reduced or suppressed by suppressing production of IL-17 cytokine.

Furthermore, the present disclosure may provide a method for activating regulatory T cells (Treg), the method comprising treating the regulatory T cells with a rebamipide compound or its salt in vitro. In the regulatory T cells activated according to the present disclosure, expression of Foxp3 may be increased.

In the method for reducing or suppressing differentiation of non-differentiated T cells into Th17 cells in vitro and the method for activating regulatory T cells according to the present disclosure, a method of treating cells with a rebamipide compound may include directly treating a culture medium for culturing the cells with the rebamipide compound or treating the culture medium with a composition containing the rebamipide compound as an active component according to the present disclosure. Further, in this case, the culture medium may be treated with the rebamipide compound to have a final concentration of 50 μM to 1500 μM.

Moreover, the composition for preventing or treating obesity according to the present disclosure shows excellent effects of reducing adipocyte, suppressing an increase in weight, and reducing total cholesterol, fatty acid glycerol, and LDL-cholesterol in the body, and also, it shows excellent effects of activating or amplifying regulatory T cells (Treg) and suppressing differentiation of Th17 cells as pathogenic cells that generate inflammatory cytokine. Further, the composition has no toxicity to drugs and no side effect, and, thus, even in the case of long-term use thereof, it is safe to take and is stable in the body.

Therefore, the present disclosure may provide a composition for food which contains a rebamipide compound or its salt as an active component and can improve or prevent symptoms of obesity. The composition for food according to the present disclosure can be readily used as a food effective in improving or preventing symptoms of obesity, such as a main ingredient or supplementary ingredient of a food, a food additive, a functional food, or beverage.

In the present disclosure, the term “food” means a natural product or a processed product containing one or more nutrients, and preferably means a directly edible product processed to a certain extent, and typically includes all of foods, food additives, functional foods, and beverages.

A food to which the composition for food according to the present disclosure can be added may include, for example, various kinds of foods, beverages, gums, teas, vitamin complex, functional foods, etc. In addition, in the present disclosure, the food may include, but may not be limited to, special nutritious foods (for example, milk formulas, infant/baby foods, or the like.), processed meat products, processed fish products, bean curd type of foods, jellied foods, noodles (for example, ramens, noodles, and the like.), bread products, dietary supplements, seasonings (for example, soy sauce, soybean paste, red pepper paste, mixed soy paste, and the like.), sauces, confectionery (for example, snacks), candies, chocolates, gums, ice creams, dairy products (for example, fermented milk, cheese, and the like.), other processed foods, kimchi, pickles (various kinds of kimchis, vegetables pickled in soy sauce, and the like.), beverages (for example, fruit juices, vegetable drinks, soybean milks, fermented beverages, and the like.), and natural seasonings (for example, ramen soup base, and the like.). The foods, beverages, or food additives can be produced by typical production methods.

Further, the term “functional food” refers to a processed food designed to sufficiently and biologically express body modulating functions relevant to biodefense rhythm control, disease prevention and recovery of a food group or a food composition added with value such that a function of a food acts and is expressed for a specific purpose by applying physical, biochemical, and biotechnological techniques to the food, and specifically, may be a health functional food. The functional food may include sitologically acceptable food supplementary additives and may further include carriers, excipients, and diluents typically used for manufacturing functional foods.

Furthermore, in the present disclosure, the term “beverage” is a generic term for drinks for quenching thirst or enjoying the taste and includes functional beverage. The beverage contains the composition for improving or preventing symptoms of obesity as an essential component at a suggested ratio and may contain other components without specific limitation, and may further contain various flavoring agents or natural carbohydrates as additional components like typical beverage.

Moreover, in addition to those described above, the composition for food for improving or preventing symptoms of obesity according to the present disclosure may contain various kinds of nutritional supplements, vitamins, minerals (electrolytes), flavoring agents such as synthesized flavoring agents and natural flavoring agents, colorings and fillers (cheese, chocolate, or the like.), pectic acid and its salt, alginic acid and its salt, organic acids, protective colloidal thickeners, pH regulating agents, stabilizers, preservatives, glycerin, alcohols, and carbonizing agents used for carbonated drinks. Such components may be used independently or in combination.

In the food containing the composition for food of the present disclosure, the composition according to the present disclosure may be contained in an amount of 0.001 wt. % to 90 wt. % and preferably 0.1 wt. % to 40 wt. % with respect to the total weight of the food. In the case of beverage, the composition may be contained in an amount of 0.001 g to 2 g and preferably 0.01 g to 0.1 g based on 100 ml. In the case of long-term intake for the purpose of health and hygiene or for the purpose of health control, an amount of the composition may be less than the above-described range. Since an active component has no problem in terms of safety, it can be used more than the above-described range and may not be limited to the above-described range.

Hereinafter, the present disclosure will be explained in detail with reference to the following examples. These examples are provided only for illustrating the present disclosure. It is obvious for one of ordinary skill in the art that the scope of the present disclosure is not limited to the following examples.

### Example 1

### Analysis of Weight Reduction Effect of Rebamipide

The inventors of the present disclosure prepared animal models induced with obesity in order to check whether rebamipide has an effect of treating obesity. As a test animal, a C57BL/6(H-2kb) mouse was used. In order to prepare animal models induced with obesity, a high fat food was fed to the animal models induced with obesity. After rebamipide was orally administered with a dose of 300 mg/kg to the animal models induced with obesity, weights of a rebamipide-treated group and a control group induced with obesity were measured.

According to a result of the measurement, the group administered with rebamipide was statistically significantly decreased as compared with the control group (refer to FIG. 1A).

Further, in order to carry out an in vitro test, at the time when an obesity index is significantly different (on the 51st day), each animal was killed, and an amount of fat in the spleen and the blood, and a treatment effect of rebamipide were investigated.

As a result of observation of the livers and the bodies of the killed mice with the naked eye, the liver of the mouse treated with rebamipide was bright red like a liver of a normal mouse, whereas the control group induced with obesity has a yellow color by accumulation of fat and was also obese (refer to FIG. 1B).

### Example 2

### Analysis of Mast Cell Reduction Effect of Rebamipide

In order to check fat inhibition effect of rebamipide, after supply of a high fat diet for 51 days was ended, a food was stopped at 12 hours before an experiment period was ended. Then, a frozen section (7 μm) obtained from a liver was haematoxylin and eosin staining and Oil red O staining. As a result of staining including the haematoxylin and eosin staining and Oil red O staining, it could be seen that in the liver obtained after oral administration with rebamipide, adipocyte were reduced as compared with a control induced with obesity (refer to FIG. 2).

### Example 3

### Cholesterol, Glucose, and Triglyceride Reduction Effect of Rebamipide in Body

Further, an experimental animal group was etherized and blood was collected from the heart. The collected blood was put into a centrifuge tube and centrifuged at 3000 rpm for 20 minutes, so that the serum was separated and kept in a freezer until it was analyzed at −70° C.

Contents of glucose, triglyceride, total cholesterol, HDL-cholesterol, and LDL-cholesterol in the serum were analyzed using an autoanalyzer (Kuadro, Italy).

As a result thereof, in the serum of the mouse administered with rebamipide, and it was confirmed that glucose, triglyceride, total cholesterol, and LDL-cholesterol are statistically significantly reduced and a concentration of HDL-cholesterol useful in the body was slightly increased (refer to FIG. 3).

### Example 4

Th17 Cell Inhibition Effect of Rebamipide in Animal Model Induced with Obesity

In order to stimulate CD4+ T cells 1×10 separated from the spleen under differentiation conditions of Th17 cells, the CD4+ T cells 1×10 were divided into a 24-well plate coated with 1 μg/Ml of an anti-CD3 antibody and treated with all of 1 μg/mL of an anti-CD28 antibody, 2 ng/ml of TGF-β, 20 ng/ml of IL-6, 10 ng/ml of anti-IL-4, and 10 ng/ml of anti-IFNr together suitable for stimulating Th17 cells. Then, the cells were stimulated for 1 hour. In this case, the cells were pre-treated with a rebamipide compound at a concentration of 1000 μM and cultured for 1 hour. The collected cells were allowed to react with a PerCP-anti-mouse CD4 antibody and a PE-anti-mouse phospho STATS antibody at 4° C. for 30 minutes in a dark field and washed with FACs buffer (0.002% sodium azide, 0.2% BSA/PBS). These cells were washed twice with Perm Wash buffer and re-floated on the FACs buffer and then analyzed with a flow cytometer.

As a result of flow cytometry conducted to check expression of Th17 cells by rebamipide in the blood and the spleen, it could be seen that in all the groups treated with rebamipide, Th17 cells were significantly reduced in both the blood and the spleen (refer to FIGS. 4A and 4B).

### Example 5

### Treg Cell Increase Effect of Rebamipide in Animal Model Induced with Obesity

In order to investigate whether or not rebamipide according to the present disclosure affected Th17 and Treg, the inventors of the present disclosure embedded a frozen section (optimal cutting temperature compound: O.C.T. compound) using the spleen of a mouse and rapidly cooled the tissue in liquid nitrogen and attached the tissue on a slide to a thickness of 7 μm using a cryo microtome. Then, the section was fixed with acetone and coated with 10% normal goat serum and blocked from a non-specific reaction for 30 minutes. Thereafter, the section was reacted at 4° C. overnight with primary antibodies, i.e. FITC-labeled anti-mFoxp3 Ab, PElabeled anti-mCD4 Ab, and APC-labeled anti-CD25 Ab, diluted with PBS (pH 7.5) at a ratio of 1:100 for Treg analysis and reacted with FITC-labeled anti-mCD4 Ab, and PE-labeled anti-mIL-17 for Th17 cell analysis. On the next day, the section was washed with a PBS solution, and a stained tissue was analyzed with a confocal microscope.

As a result thereof, as illustrated in FIG. 5A and FIG. 5B, in the group treated with rebamipide, expression of Th17 was reduced but Treg cells were increased.

### Example 6

### Analysis of Obesity Improvement Effect Depending on Treatment Concentration of Rebamipide

<6-1> Analysis of Weight Reduction Effect

In order to check an obesity improvement effect depending on a treatment concentration of rebamipide, the inventors of the present disclosure orally administered rebamipide with a dose of 30 mg/kg and 100 mg/kg to an animal models induced with obesity prepared in Example 1 and then measured weights of the rebamipide treatment group and a mouse induced with obesity as a control group.

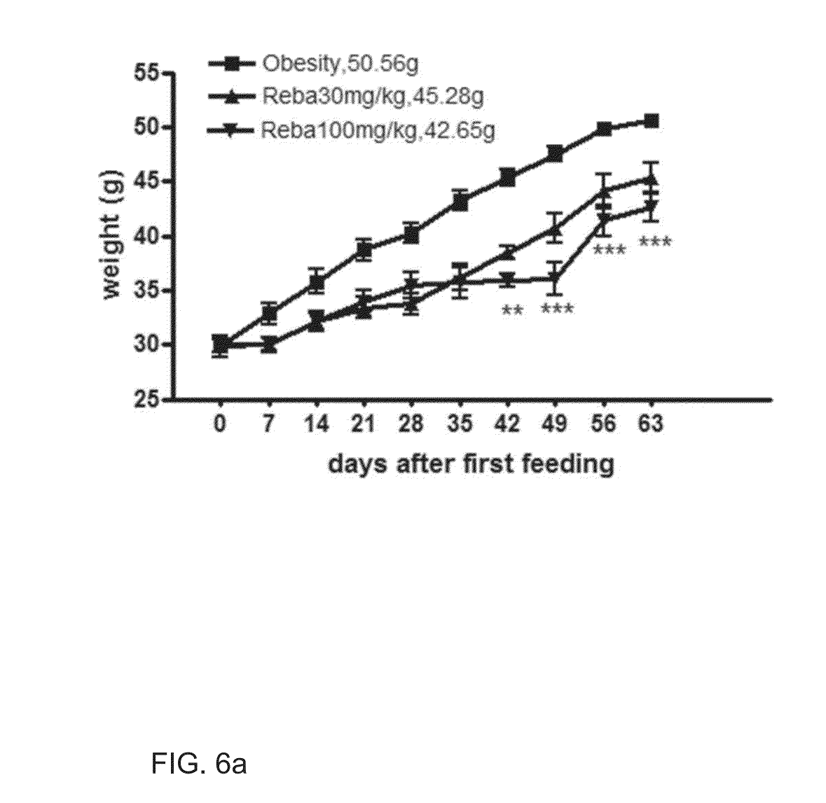

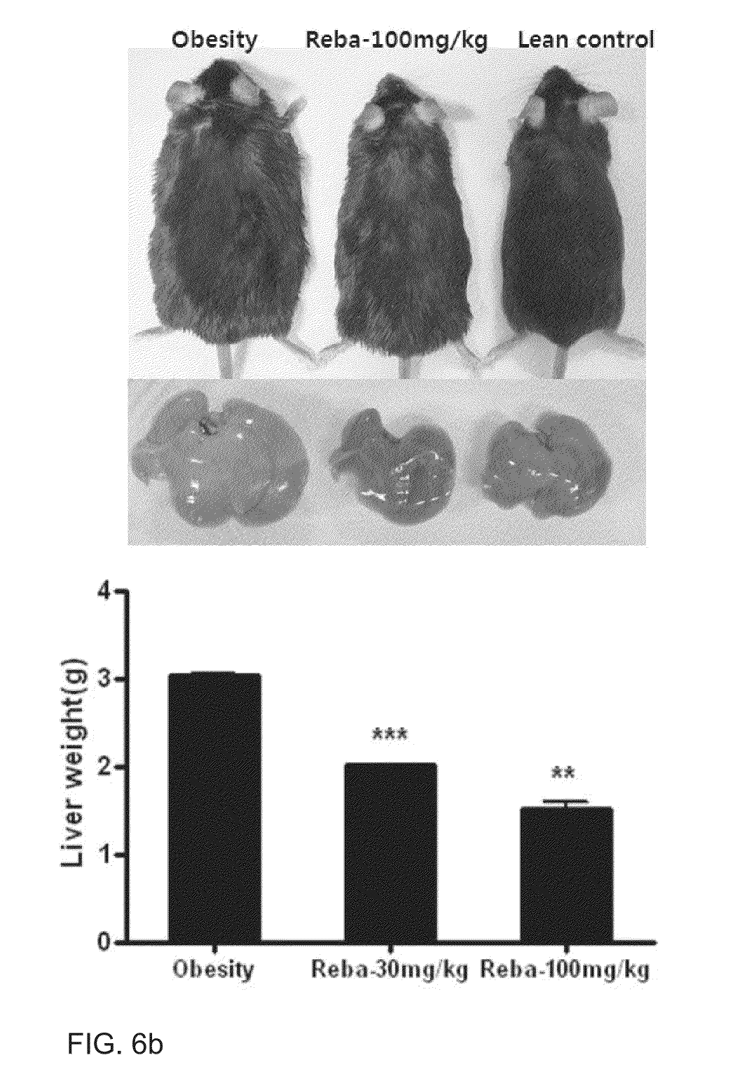

As a result of the measurement, it could be seen that a weight of the group orally administered with rebamipide was statistically reduced as compared with the control group, and it could be seen that in the case of administration with a dose of 100 mg/kg, a weight reduction effect was highest. Further, it could be seen that even when rebamipide of the present disclosure was used with a small dose of 30 mg/kg, a weight reduction effect was illustrated (refer to FIG. 6A).

For in vitro test, on the 63rd day after treatment with rebamipide, an experimental animal was killed and the liver of the mouse was extracted, and then, a size of the mouse and a weight of the liver were measured with the naked eye. As a result of the measurement, it could be seen that the liver of the mouse induced with obesity was increased and became fatty liver tinged with yellow, whereas the liver of the mouse orally administered with a dose of 100 mg/kg of rebamipide was bright red similarly to a liver of a normal control mouse (refer to FIG. 6B).

<6-2> Internal Cholesterol, Glucose, and Triglyceride Reduction Effect of Rebamipide

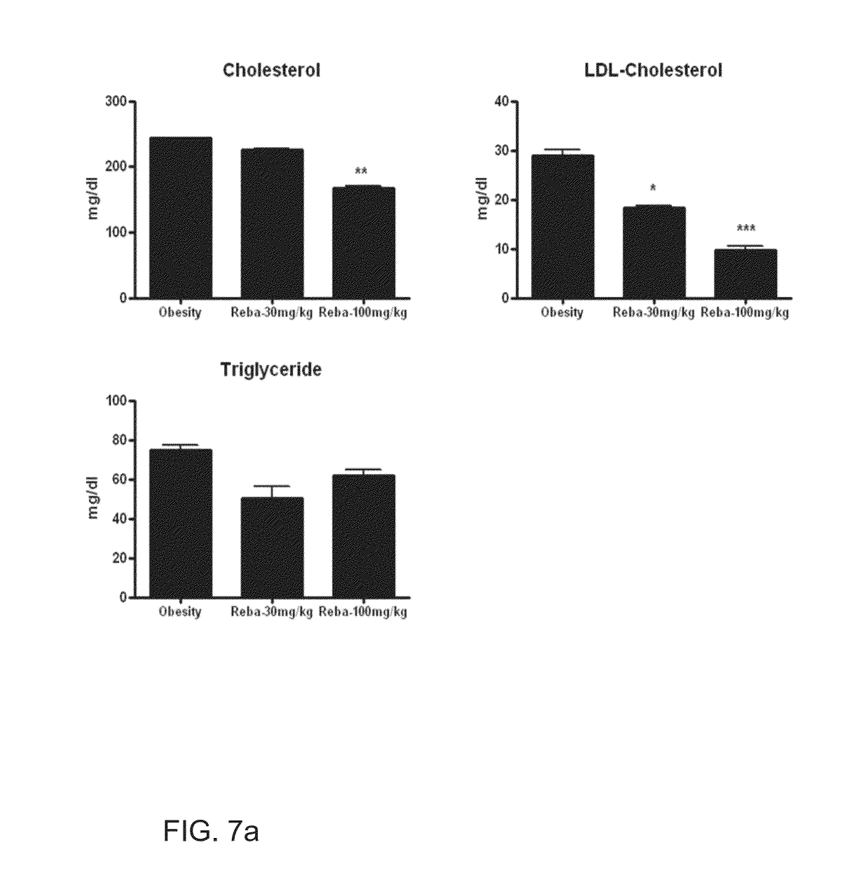

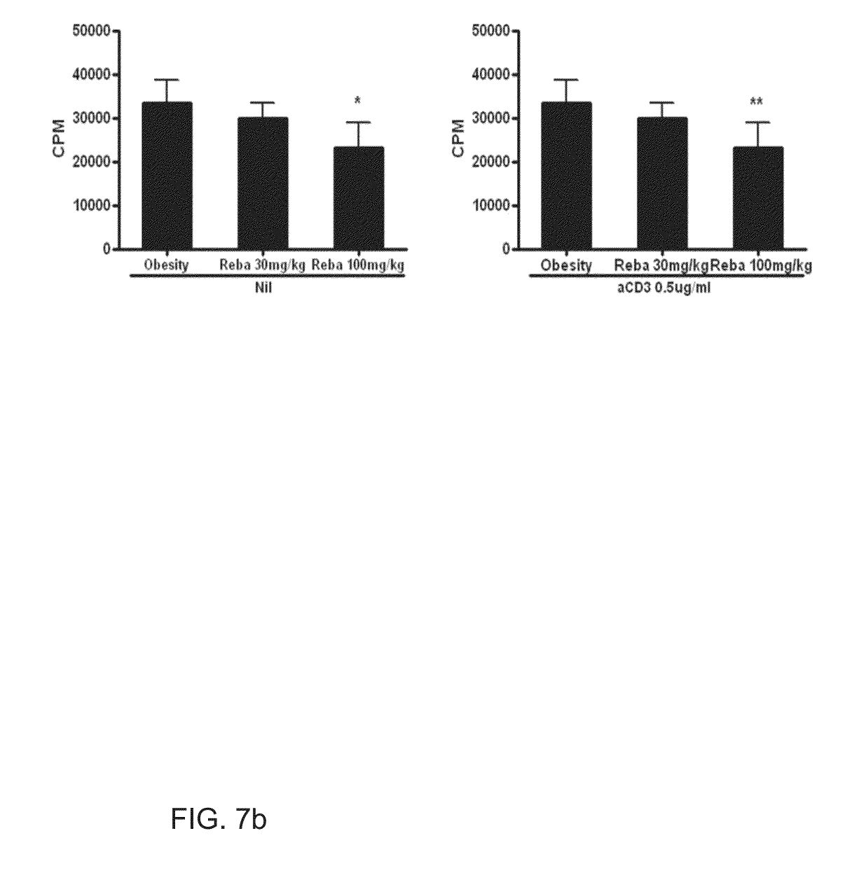

An experimental animal group was etherized and blood was collected from the heart. The collected blood was put into a centrifuge tube and centrifuged at 8000 rpm for 8 minutes at 20° C., so that the serum was separated. Concentrations of cholesterol, LDL-cholesterol, and triglyceride in the serum were requested from a contract research organization, and as a result thereof, it could be seen that in the group orally administered with rebamipide, concentrations of cholesterol, LDL-cholesterol, and triglyceride was statistically significantly reduced, and particularly, in the case of treatment with rebamipide with a dose of 100 mg/kg, such an effect was generally highest (refer to FIG. 7A).

The spleen was extracted from the mouse of each group and spleen cells 2×10⁵ of each mouse were divided into a 96-well plate and stimulated with 0.5 μg/ml of aCD3 and then cultured for 3 days. 16 hours before acquirement, the cells were stimulated with thymidine of 1 μM/well, and then, 16 hours after stimulation, proliferation of the mouse cells was measured. As a result of the measurement, it could be seen that in the group orally administered with rebamipide of the present disclosure, inhibition of cell proliferation was decreased, and particularly, in the case of treatment with rebamipide with a dose of 100 mg/kg, such an effect was higher (refer to FIG. 7B).

### Example 7

### Analysis of Mast Cell Inhibition Effect of Rebamipide

<7-1> Oil Red O Analysis

In order to check whether or not rebamipide has a mast cell inhibition effect, the inventors of the present disclosure divided pre-adipocyte in an amount of 1.2×10⁴/2 ml into a 24-well plate, and when the cells were completely packed, the cells were cultured again for 48 hours. After 48 hours, differentiation of adipocyte was induced with replacement of a culture medium added with dexamethasone, insulin, and isobutylmethylxanthine. In this case, rebamipide was also used to stimulate while being dissolved in DMSO.

3 days after differentiation, differentiation of adipocyte was maintained with replacement with a culture medium added with dexamethasone and insulin once per two days. After 4 days, Oil red O analysis was carried out.

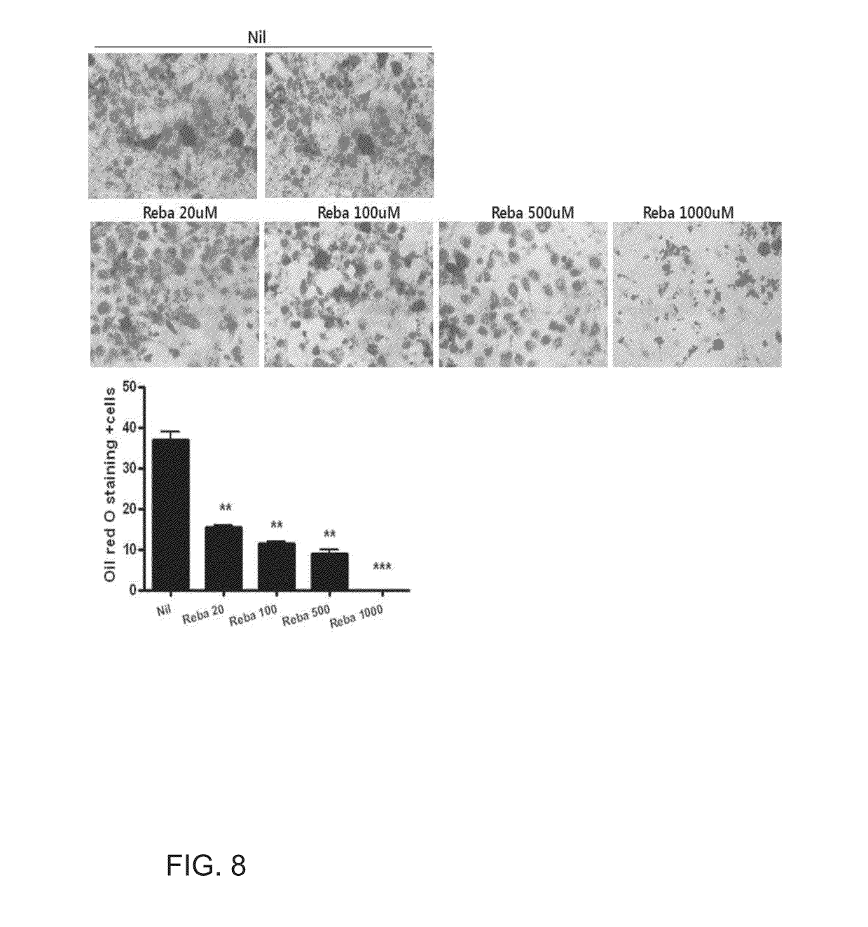

As a result of the analysis, it could be seen that differentiation of adipocyte did not occur in the cells treated with rebamipide as compared with the cells which were not treated with rebamipide, which was dependent on dose of rebamipide (refer to FIG. 8).

<7-2> PCR Analysis

The inventors of the present disclosure differentiated adipocyte by the method of Example <7-1>. 3 days after differentiation, differentiation of adipocyte was maintained with replacement with a culture medium added with dexamethasone and insulin once per two days, and after 4 days, the cells were treated with trizol and RNA was separated therefrom and cDNA was synthesized. Then, by real time PCR, mRNA expression of c/EBP, adiponectin, Leptin aP2, GLTU, and Cytochrome C1 as proadipogenic transcription factors was observed.

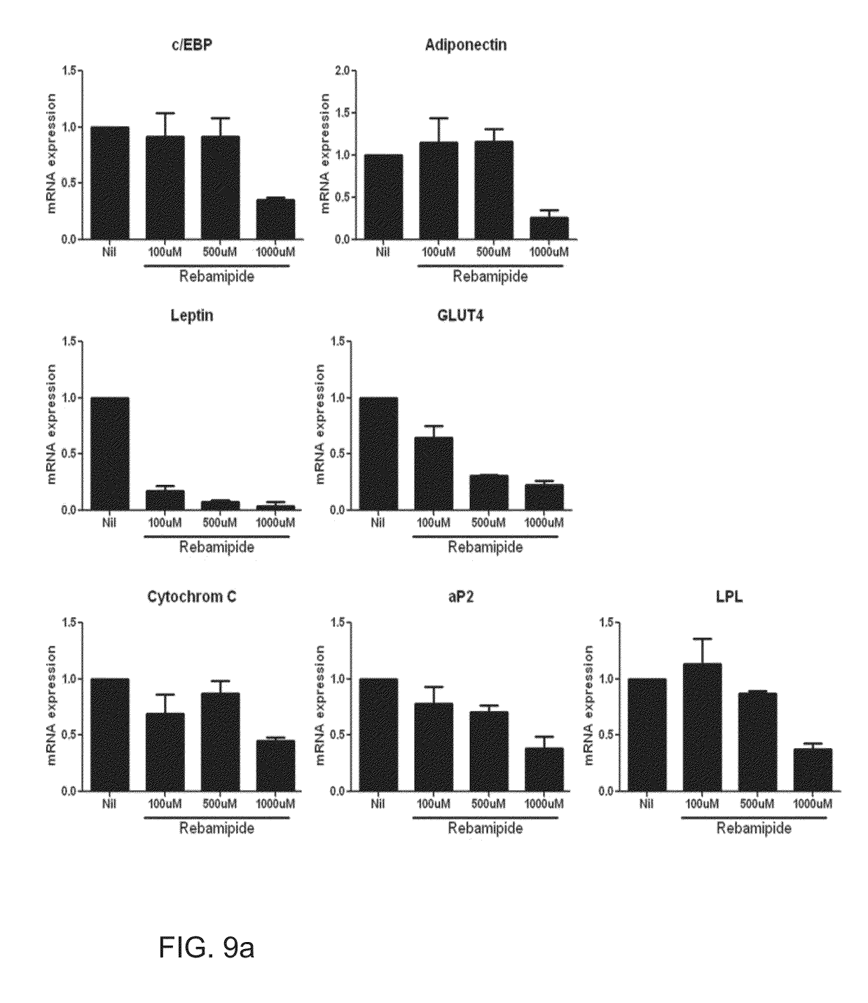

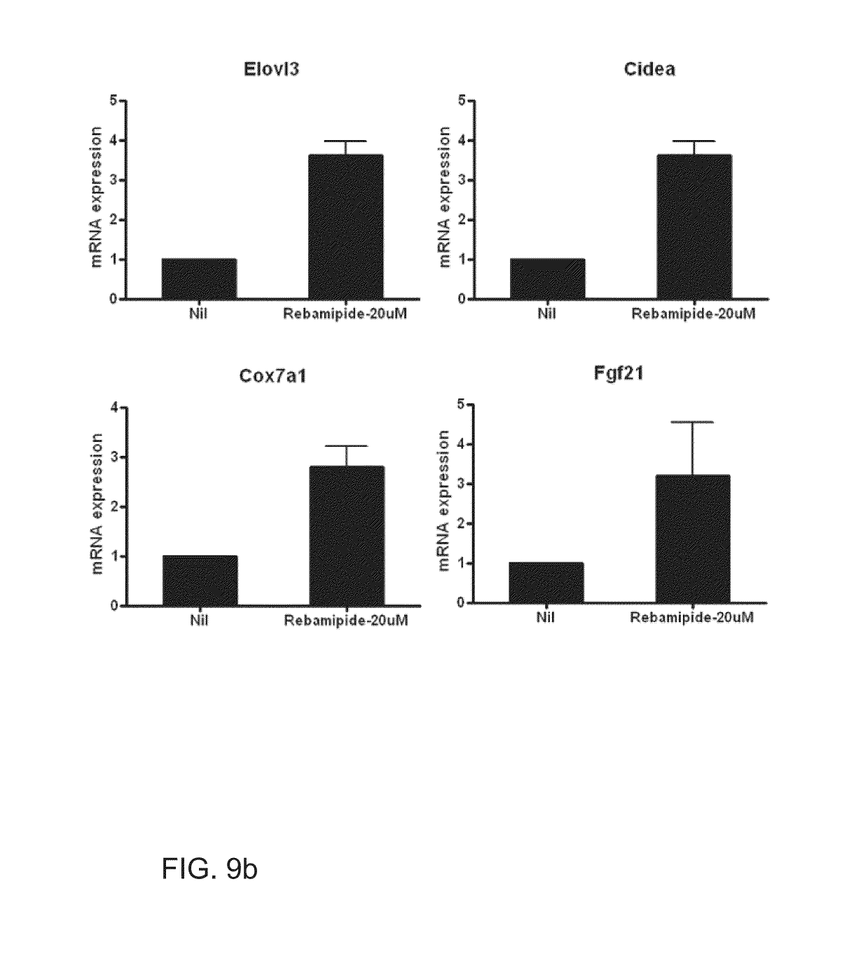

As a result thereof, it could be seen that mRNA expression of c/EBP, adiponectin, Leptin aP2, GLTU, and Cytochrome C1 as cytokines relevant to mass cells was reduced depending on a concentration of rebamipide. Further, as a result of observation of Elov13, Cidea, Cox7a1, Cidea, and Fgf21as molecules relevant to brown fat genes by real time PCR, it was observed that in a well treated with 20 μm of rebamipide, a level of genes relevant to brown fat was increased. Such a result suggested that rebamipide converted white fat into brown fat (refer to FIG. 9).

Therefore, it was confirmed that rebamipide of the present disclosure was effective in inhibition of mast cells and capable of converting white fat into brown fat good for the body.

Table 1
Primer sequence

| Primer | Primer sequence |
|---|---|
| C/EBP-a | 5′-CAAGAACAGCAACGAGTACCG-3′ |
| (forward) | (SEQ ID NO: 1) |
| C/EBP-a | 5′-GTCACTGGTCAACTCCAGCAC-3′ |
| (reverse) | (SEQ ID NO: 2) |
| Adiponectin | 5′-GTCAGTGGATCTGACGACACCAA-3′ |
| (forward) | (SEQ ID NO: 3) |
| Adiponectin | 5′-ATGCCTGCCATCCAACCTG-3′ |
| (reverse) | (SEQ ID NO: 4) |
| Leptin | 5′-CCTCATCAAGACCATTGTCACC-3′ |
| (forward) | (SEQ ID NO: 5) |
| Leptin | 5′-TCTCCAGGTCATTGGCTATCTG-3′ |
| (reverse) | (SEQ ID NO: 6) |
| GLTU4 | 5′-ACTCTTGCCACACAGGCTCT-3′ |
| (forward) | (SEQ ID NO: 7) |
| GLTU4 | 5′-AATGGAGACTGATGCGCTCT-3′ |
| (reverse) | (SEQ ID NO: 8) |
| Cytochrome c1 | 5′-GCTACCCATGGTCTCATCGT-3′ |
| (forward) | (SEQ ID NO: 9) |
| Cytochrome c1 | 5′-CATCATCATTAGGGCCATCC-3′ |
| (reverse) | (SEQ ID NO: 10) |
| aP2 | 5′-GATGCCTTTGTGGGAACCT-3′ |
| (forward) | (SEQ ID NO: 11) |
| aP2 | 5′-CTGTCGTCTGCGGTGATTT-3′ |
| (reverse) | (SEQ ID NO: 12) |
| LPL | 5′-GGAAGAGATTTCTCAGACATCG-3′ |
| (forward) | (SEQ ID NO: 13) |
| LPL | 5′-CTACAATGACATTGGAGTCAGG-3′ |
| (reverse) | (SEQ ID NO: 14) |
| Elovl3 | 5′-CGGGTTAAAAATGGACCTGA-3′ |
| (forward) | (SEQ ID NO: 15) |
| Elovl3 | 5′-CCAACAACGATGAGCAACAG-3′ |
| (reverse) | (SEQ ID NO: 16) |
| Cidea | 5′-GCCGTGTTAAGGAATCTGCTG-3′ |
| (forward) | (SEQ ID NO: 17) |
| Cidea | 5′-TGCTCTTCTGTATCGCCCAGT-3′ |
| (reverse) | (SEQ ID NO: 18) |
| Cox7a1 | 5′-AGAAAACCGTGTGGCAGAGA-3′ |
| (forward) | (SEQ ID NO: 19) |
| Cox7a1 | 5′-CAGCGTCATGGTCAGTCTGT-3′ |
| (reverse) | (SEQ ID NO: 20) |
| Fgf21 | 5′-CCTCTAGGTTTCTTTGCCAACAG-3′ |
| (forward) | (SEQ ID NO: 21) |
| Fgf21 | 5′-AAGCTGCAGGCCTCAGGAT-3′ |
| (reverse) | (SEQ ID NO: 22) |

### Example 8

### Comparative Analysis Between Brown Fat and White Fat after Treatment with Rebamipide

Through the above-described experiment, the inventors of the present disclosure found that rebamipide of the present disclosure was effective in preventing and suppressing obesity, and further conducted another experiment in order to check an effect of rebamipide on formation of white fat and brown fat in vivo. Meanwhile, white fat stores excessive energy in the form of triglyceride in the body. Therefore, when energy excess occurs due to lack of exercise or overeating, the number of white fat cells is increased and obesity is induced and the body becomes apt to gain weight accordingly. However, brown fat consumes stored energy in the form of heat, and, thus, it does not cause weight increase or obesity.

Thus, in order to check an effect of treatment with rebamipide on white fat and brown fat in vivo, the inventors of the present disclosure separated fat from an interscapular region of an animal group induced with obesity and a group treated with rebamipide with a dose of 30 mg/kg and 100 mg/kg and divided into white fat and brown fat. A weight of each of the white fat and the brown fat was measured, and an amount of the brown fat with respect to the white fat was calculated.

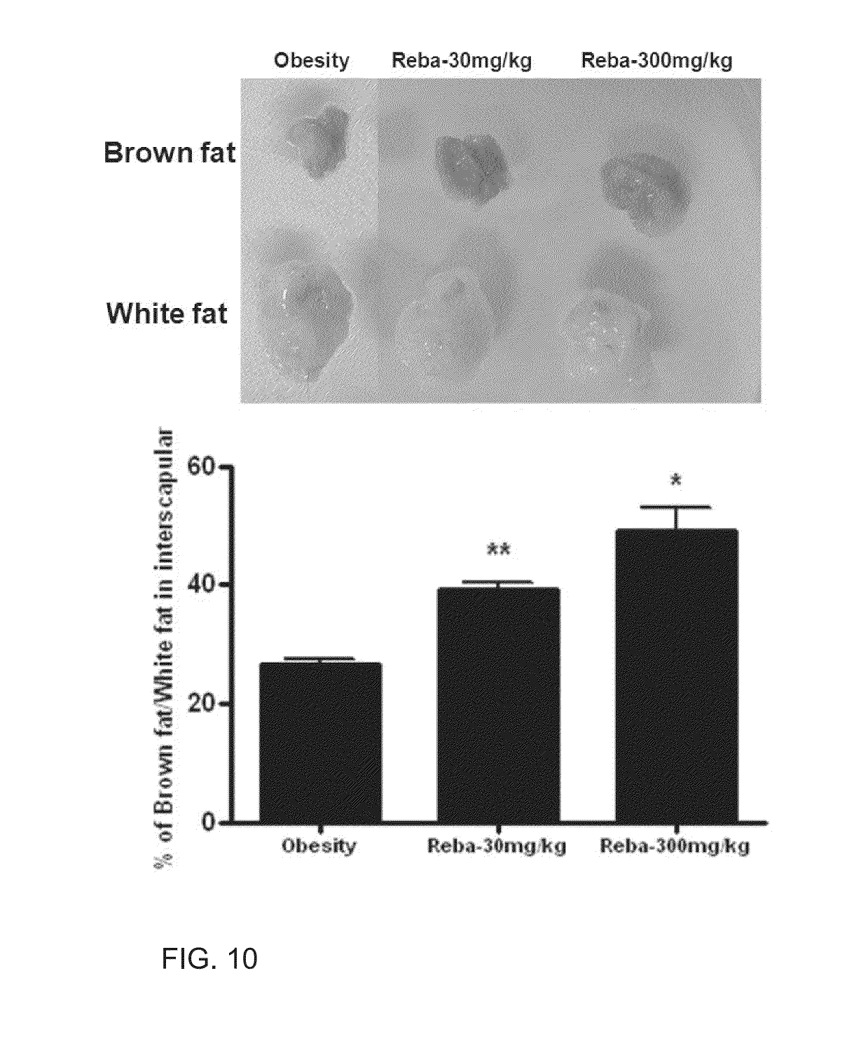

As a result thereof, it could be seen that a ratio of the brown fat to the white fat was increased in the animal group treated with rebamipide as compared with the animal group induced with obesity, so that rebamipide of the present disclosure helped in forming brown fat good for the body (refer to FIG. 10).

### Example 9

### Analysis of Treatment Effect of Obesity Induced Arthritis after Treatment with Rebamipide

<9-1> Arthritis Index Evaluation

Many studies have already disclosed that obesity is a cause of various adult diseases such as diabetes or arteriosclerosis and also has a bad influence on arthritis. Therefore, the inventors of the present disclosure confirmed that rebamipide has an obesity inhibition effect, and then, induced obesity of a 4-week-old C57BL/6 mouse with high fat diet of 60 kcal in order to check whether rebamipide also has an effect on treatment of obesity induced arthritis. When a weight of the mouse was 30 g, arthritis was induced. In order to prepare an animal model with arthritis, Type 2 collagen (Cll) was dissolved in a 0.1 N acetic acid solution to be 2 mg/ml and dialyzed with a dialysis buffer (50 mM Tris, 0.2 N NaCl) and then equivalently mixed with a complete Freund's adjuvant (CFA, Chondrex) containing M. tuberculosis. The mixture was hypodermically given to a base of a tail of the mouse, and, thus, an immunogen of 100 μl (that is, 100 μl/100 μg) was injected to each mouse (first injection). After 2 weeks, the same Cll was equivalently mixed with incomplete Freud's adjuvant (IFA, Chondrex) and 100 μm (that is, 100 μl/100 μg) of the mixture was injected to one hind leg (foot pad) (second injection).

After second immunization through the second injection, 300 mg/kg of rebamipide was orally injected 10 times to a mouth three times per week. Herein, the rebamipide was used while being dissolved in a 0.5% CMC solution. Each group included 5 mice, and evaluation of arthritis was carried out for up to 61 days. Further, for in vitro test, each animal was killed when there was a significant difference in arthritis index, and as described below, activity of arthritis in blood and articular tissues and a treatment effect of rebamipide were studied.

3 weeks after the first injection as a starting point, four observers who did not know about the experiment evaluated impression-severity of arthritis twice per week and observed for up to 61 days. In the evaluation of arthritis, there were used an average value obtained by uniting scores on the following scale in three legs of each animal except a leg administered with Cll/CFA during the second injection and dividing the sum by 3, and an average value obtained by uniting values obtained by three observers from each animal model and dividing the sum. Scores and standards used for evaluation of arthritis were as follows.

—Evaluation Standard—

Score 0: No edema or swelling

Score 1: Slight edema and rubefaction limited to foot or ankle joint

Score 2: Slight edema and rubefaction ranging from ankle joint to metatarsal

Score 3: Moderate edema and rubefaction ranging from ankle joint to metatarsal

Score 4: Edema and rubefaction ranging from ankle to a full leg

Since the maximum arthritis index of each animal is 4, the highest disease index of each mouse is 16.

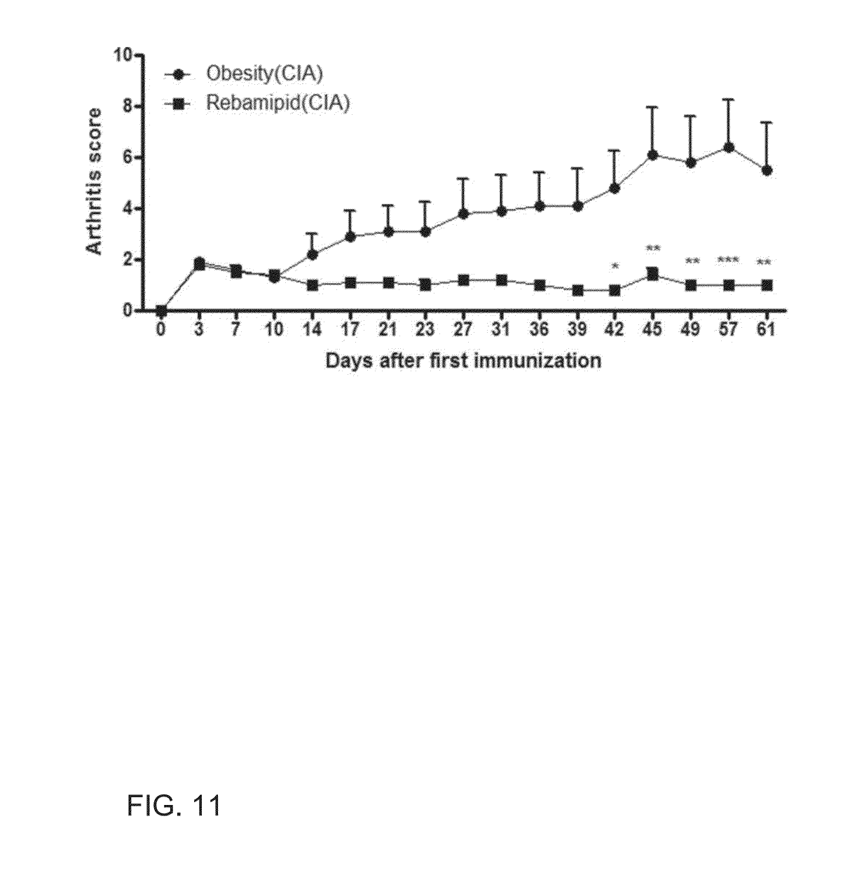

As a result of the observation, it was illustrated that in the mouse model with collagen induced arthritis, an arthritis index was continuously increased after 2 weeks and arthritis was worsen, whereas in the animal model injected with rebamipide, an arthritis index was decreased (refer to FIG. 11).

<9-2> Histological Inspection

Each animal model with collagen induced arthritis (CIA) was orally administered with rebamipide with a dose of 300 mg/kg. After 61 days, euthanasia was administered to the experimental animals. Then, hind legs of the mouse were fixed with 10% formalin, and compounds of calcium were removed from the bone and paraffin was coated. A section of the joint (7 μm) was prepared and stained with hematoxylin and eosin. Further, in order to check damage of cartilage, a histological inspection was carried out by staining with Toluidine blue and safranin O.

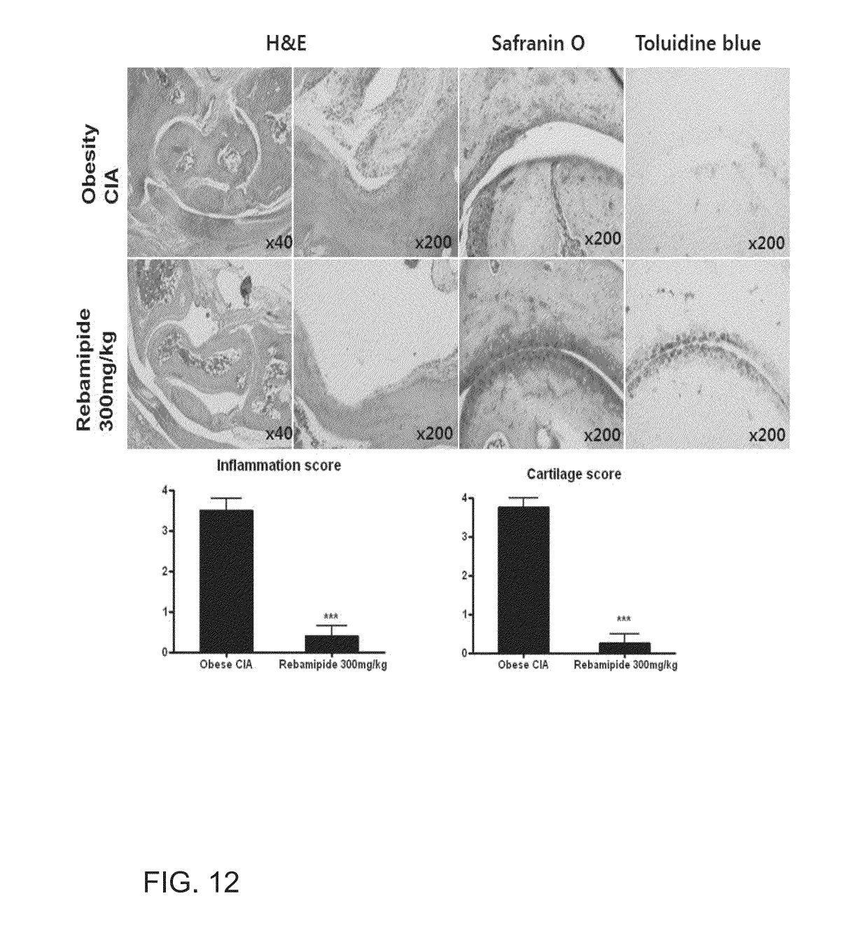

As a result of the histological inspection, the joint of the CIA animal was infiltrated by immune cells and formation of pannus, damage of cartilage, and erosion of bones were observed. Meanwhile, it was observed that in the animal orally administered with rebamipide with a dose of 300 mg/kg, damage of the joint and cartilage was not severe and maintained similarly to a normal mouse (refer to FIG. 12).

<9-3> Analysis by ELISA

Blood was collected from the heart of a mouse in each group and centrifuged at 8000 rpm for 8 minutes at 20° C., so that the mouse serum of each group was obtained from a supernatant of the blood. The serum was diluted at a ratio of 1:100, and type-II-collagen-specific IgG and IgG2a were measured by an ELISA method.

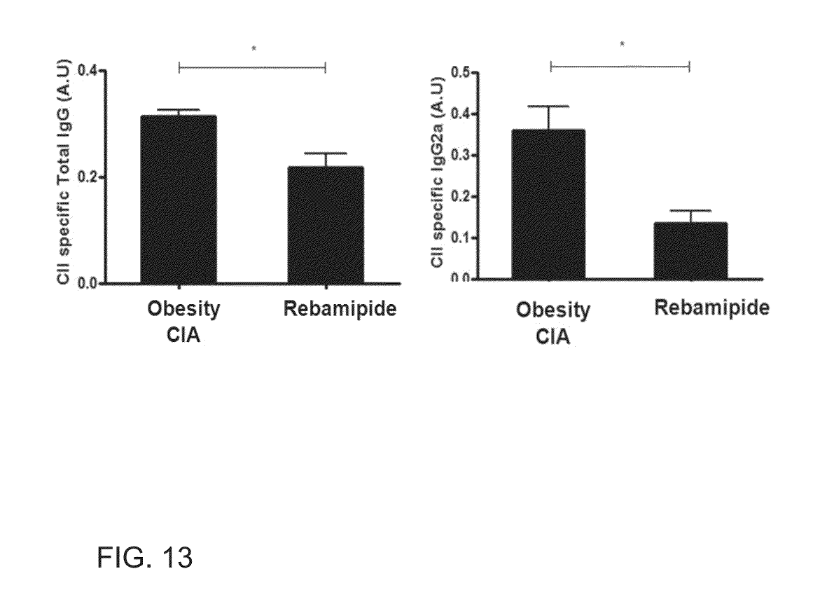

As a result thereof, it could be seen that the measurement values of IgG and IgG2a were significantly decreased in the rebamipide treatment group as compared with the animal group with obesity induced arthritis (refer to FIG. 13).

<9-4> Immunohistochemistry Staining Analysis

In order to check whether or not rebamipide of the present disclosure specifically can inhibit inflammatory cytokines such as IL-17, IL-6, IL-1b, TNF-a, and Nitrothyrosine in the joint of an animal model with obesity induced arthritis, the joint of a mouse was treated by the same method as illustrated in Example <9-2>. Then, inflammatory cytokines were stained by with an immunochemical staining method and analyzed with an optical microscope.

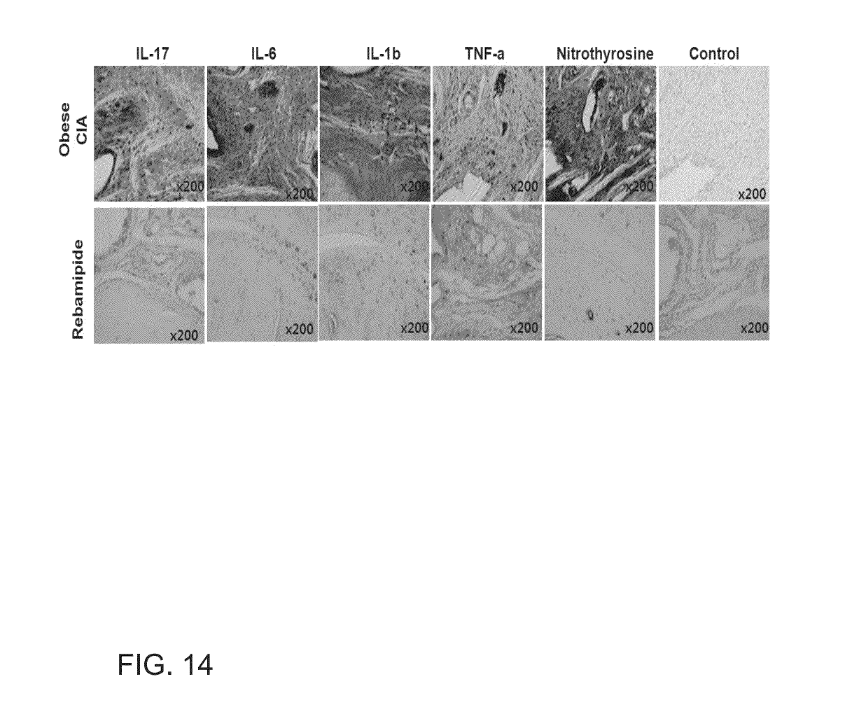

As a result thereof, it could be seen that infiltration of IL-17, IL-6, IL-1b, TNF-a, and Nitrothyrosine as inflammatory cytokines was inhibited in the rebamipide treatment group as compared with the animal group with obesity induced arthritis (refer to FIG. 14).

<9-5> Analysis of Th17 Cell Inhibition and Treg Cell Increase Effect of Rebamipide in Animal Model with Obesity Induced Arthritis

Further, in order to investigate whether or not rebamipide affects Th17 and Treg, the inventors of the present disclosure embedded a frozen section (optimal cutting temperature compound: O.C.T. compound) using the spleen of a mouse and rapidly cooled the tissue in liquid nitrogen and attached the tissue on a slide to a thickness of 7 μm using a cryo microtome. Then, the section was fixed with acetone and coated with 10% normal goat serum and blocked from a non-specific reaction for 30 minutes. Thereafter, the section was reacted at 4° C. overnight with primary antibodies, that is, FITC-labeled anti-mFoxp3 Ab, PElabeled anti-mCD4 Ab, and APC-labeled anti-CD25 Ab, diluted with PBS (pH 7.5) at a ratio of 1:100 for Treg analysis and reacted with FITC-labeled anti-mCD4 Ab, and PE-labeled anti-mIL-17 for Th17 cell analysis. On the next day, the section was washed with a PBS solution, and a stained tissue was analyzed with a confocal microscope.

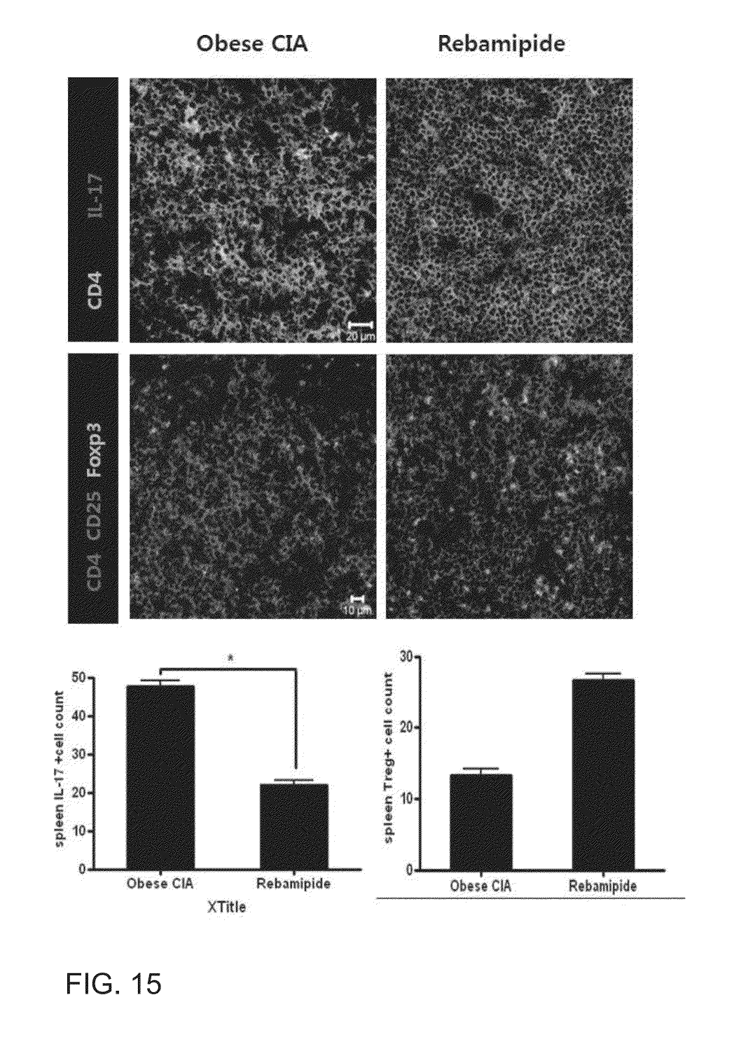

As a result thereof, as illustrated in FIG. 15, in the rebamipide treatment group, expression of Th17 was reduced but Treg cells were increased.

<9-6> STAT3 Inhibition Effect of Rebamipide in Animal Model with Obesity Induced Arthritis

In order to investigate whether or not rebamipide can inhibit STAT3, the inventors of the present disclosure extracted the spleen from a mouse in each group, and embedded a frozen section (optimal cutting temperature compound: O.C.T. compound) using the extracted spleen and rapidly cooled the tissue in liquid nitrogen and attached the tissue on a slide to a thickness of 7 μm using a cryo microtome. Then, the section was fixed with acetone and coated with 10% normal goat serum and blocked from a non-specific reaction for 30 minutes. Thereafter, the section was reacted at 4° C. overnight with primary antibodies, that is, FITC-labeled anti-mFoxp3 Ab, FITC labeled anti-mCD4 Ab, PE-labeled anti-p-STAT3 s727 Ab, and PE-labeled anti-p-STAT3 y705 Ab, diluted with PBS (pH 7.5) at a ratio of 1:50 for analyzing STAT3. On the next day, the section was washed with a PBS solution, and a stained tissue was analyzed with a confocal microscope.

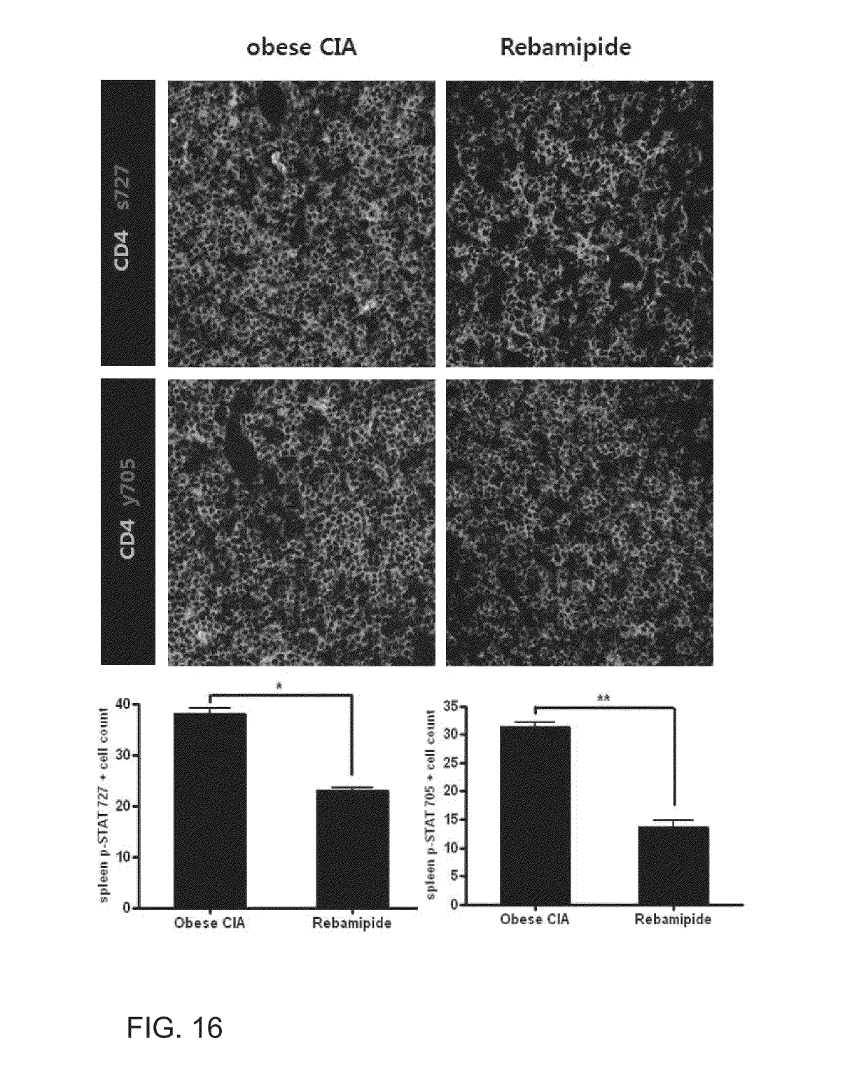

As a result thereof, it could be seen that p-STAT3 s727 and p-STAT3 s705 were decreased in the rebamipide treatment group as compared with the group with obesity induced arthritis (refer to FIG. 16).

In conclusion, from the above-described results, the inventors of the present disclosure found that rebamipide compound of the present disclosure was highly effective in reducing adipocyte and reducing a total cholesterol content and highly effective in suppressing differentiation of cytotoxic Th17 cells that generate and secretes inflammatory cytokine, and also effective in improving activity of regulatory T cells (Treg) capable of suppressing a function of abnormally activated immune cells and controlling an inflammatory reaction, and, thus, it is possible to prevent and treat obesity.

Further, the rebamipide compound of the present disclosure has an effect of improving obesity even with a small dose and exhibits an excellent pharmacological effect and is effective in forming brown fat good for the body. Furthermore, the rebamipide compound of the present disclosure is also effective in preventing and treating severe rheumatoid arthritis induced by obesity.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A method for preventing or treating obesity in a subject comprising administering to the subject a composition comprising a rebamipide compound or its pharmaceutically acceptable salt as an active component.

2. The method of claim 1, wherein the composition comprises rebamipide in a concentration of 10 mg/kg to 1000 mg/kg body weight.

3. A method for preventing or treating a complication caused by obesity in a subject, wherein the complication is selected from the group consisting of intra-abdominal fat syndrome, metabolic syndrome, hypertriglyceridemia, hypo-HDL cholesterolemia, angina, myocardial infarction, ostarthritis, cancers related to weight gain, orthostatic hypotension, pulmonary hypertension, diabetes, hypertension, damaged glucose tolerance, coronary thrombosis, atherosclerosis, gall bladder disease, insulin resistant diabetes, chronic total occlusion, thromboembolism, heart disease, lipid syndrome, and hyperglycemia, comprising administering to the subject a composition comprising a rebamipide compound or its pharmaceutically acceptable salt as an active component.

4. The method according to claim 3, wherein the ball bladder disease is cholelithiasis.

5. The method of claim 3, wherein the composition comprises rebamipide at a concentration of 10 mg/kg to 1000 mg/kg body weight.

6. A method for reducing body weight in a subject, comprising administering to the subject a composition comprising a rebamipide compound or its pharmaceutically acceptable salt as an active component.

7. The method of claim 6, wherein the composition comprises rebamipide at a concentration of 10 mg/kg to 1000 mg/kg body weight.


SEQUENCE LISTING (22 sequences)

SEQ ID NO: 1 - DNA, 21 bp, Artificial Sequence
  Primer - C/EBP-a(forward)
caagaacagcaacgagtaccg

SEQ ID NO: 2 - DNA, 21 bp, Artificial Sequence
  Primer - C/EBP-a(reverse)
gtcactggtcaactccagcac

SEQ ID NO: 3 - DNA, 23 bp, Artificial Sequence
  Primer - Adiponectin(forward)
gtcagtggatctgacgacaccaa

SEQ ID NO: 4 - DNA, 19 bp, Artificial Sequence
  Primer - Adiponectin(reverse)
atgcctgccatccaacctg

SEQ ID NO: 5 - DNA, 22 bp, Artificial Sequence
  Primer - Leptin(forward)
cctcatcaagaccattgtcacc

SEQ ID NO: 6 - DNA, 22 bp, Artificial Sequence
  Leptin(reverse)
tctccaggtcattggctatctg

SEQ ID NO: 7 - DNA, 20 bp, Artificial Sequence
  Primer - GLTU4(forward)
actcttgccacacaggctct

SEQ ID NO: 8 - DNA, 20 bp, Artificial Sequence
  Primer - GLTU4(reverse)
aatggagactgatgcgctct

SEQ ID NO: 9 - DNA, 20 bp, Artificial Sequence
  Primer - Cytochrome c1(forward)
gctacccatggtctcatcgt

SEQ ID NO: 10 - DNA, 20 bp, Artificial Sequence
  Cytochrome c1 (reverse)
catcatcattagggccatcc

SEQ ID NO: 11 - DNA, 19 bp, Artificial Sequence
  Primer - aP2(forward)
gatgcctttgtgggaacct

SEQ ID NO: 12 - DNA, 19 bp, Artificial Sequence
  Primer - aP2(reverse)
ctgtcgtctgcggtgattt

SEQ ID NO: 13 - DNA, 22 bp, Artificial Sequence
  Primer - LPL(forward)
ggaagagatttctcagacatcg

SEQ ID NO: 14 - DNA, 22 bp, Artificial Sequence
  Primer - LPL(reverse)
ctacaatgacattggagtcagg

SEQ ID NO: 15 - DNA, 20 bp, Artificial Sequence
  Primer - Elovl3(forward)
cgggttaaaaatggacctga

SEQ ID NO: 16 - DNA, 20 bp, Artificial Sequence
  Primer - Elovl3(reverse)
ccaacaacgatgagcaacag

SEQ ID NO: 17 - DNA, 21 bp, Artificial Sequence
  Primer - Cidea(forward)
gccgtgttaaggaatctgctg

SEQ ID NO: 18 - DNA, 21 bp, Artificial Sequence
  Primer - Cidea(reverse)
tgctcttctgtatcgcccagt

SEQ ID NO: 19 - DNA, 20 bp, Artificial Sequence
  Primer - Cox7a1(forward)
agaaaaccgtgtggcagaga

SEQ ID NO: 20 - DNA, 20 bp, Artificial Sequence
  Primer - Cox7a1(reverse)
cagcgtcatggtcagtctgt

SEQ ID NO: 21 - DNA, 23 bp, Artificial Sequence
  Primer - Fgf21(forward)
cctctaggtttctttgccaacag

SEQ ID NO: 22 - DNA, 19 bp, Artificial Sequence
  Primer  - Fgf21(reverse)
aagctgcaggcctcaggat